SEQUENCE LISTING (11 sequences)

SEQ ID NO: 1 - protein, 8 aa, Synthetic Construct
GFTFSNYA

SEQ ID NO: 2 - protein, 8 aa, Synthetic Construct
ISGGGNTY

SEQ ID NO: 3 - protein, 10 aa, Synthetic Construct
VRATRDYFDY

SEQ ID NO: 4 - protein, 12 aa, Synthetic Construct
QSLLNSRTRKSY

SEQ ID NO: 5 - skipped

SEQ ID NO: 6 - protein, 8 aa, Synthetic Construct
EQSYYLYT

SEQ ID NO: 7 - protein, 112 aa, Synthetic Construct
DIVMSQSPSSLAVSVGEKVTMSCKSSQSLLNSRTRKSYLAWYQQKPGQSPKLLIYWASTRESGVPDRFTGSGSGTDFTLTISSVQAEDLAVYSCEQSYYLYTFGGGTKLEIK

SEQ ID NO: 8 - protein, 116 aa, Synthetic Construct
EVKLVESGGGLVKPGGSLQLSCAASGFTFSNYAMSWVRQTPEKRLEWVASISGGGNTYYPDSVKGRFTISRDNARNILYLQMNSLRSEDTAMYYCVRATRDYFDYWGQGTTLTVSS

SEQ ID NO: 9 - protein, 676 aa, Synthetic Construct [sequence omitted]

SEQ ID NO: 10 - protein, 20 aa, Synthetic Construct
AEKDAVNESGRVEFGSYADA

SEQ ID NO: 11 - protein, 10 aa, Synthetic Construct
YPYDVPDYAG

(19) United States
(12) Patent Application Publication
(10) Pub. No.: US 2024/0368272 A1
(43) Pub. Date: Nov. 7, 2024

(54) COMPOSITIONS AND METHODS FOR TREATING LONG QT SYNDROME

(71) Applicant: University of Bern, Bern (CH)

(72) Inventor: Jin Li, Bern (CH)

(21) Appl. No.: 18/688,546

(22) PCT Filed: Sep. 2, 2022
(86) PCT No.: PCT/IB2022/058286

§ 371 Date: Mar. 1, 2024

Related U.S. Application Data
(60) Provisional application No. 63/240,494, filed on Sep. 3, 2021.

(51) Int. Cl. C07K 16/28; A61K 39/00; A61P 9/06; C12N 15/85

(52) U.S. Cl. CPC C07K 16/28; A61P 9/06; C12N 15/85; A61K 2039/505; C07K 2317/14; C07K 2317/565

(57) ABSTRACT

The present disclosure provides anti-KCNQ1 monoclonal antibodies and their use in treating long QT syndrome (LQTS).

### CROSS-REFERENCE TO RELATED APPLICATIONS

The present application claims the benefit of priority to U.S. Provisional Application No. 63/240,494, filed Sep. 3, 2021, the disclosure of which is incorporated herein by reference in its entirety.

### FIELD OF THE DISCLOSURE

The present application is related to materials and methods for the treatment of Long QT syndrome.

### INCORPORATION BY REFERENCE OF THE SEQUENCE LISTING

This application contains, as a separate part of disclosure, a Sequence Listing in computer-readable form (Filename: 56242A_SeqListing.XML10,587 bytes—ASCII text file created Sep. 1, 2022) which is incorporated by reference herein in its entirety.

### BACKGROUND

Long QT syndrome (LQTS) is responsible for a significant proportion of sudden cardiac deaths (1). Genetic mutations leading to a loss of function of the cardiac voltage-gated KCNH2 (LQTS type 2 [LQTS2]) or KCNQ1 (LQTS type 1 [LQTS1]) potassium ion (K⁺) channels are the most common causes (2). As a result, the corresponding repolarizing currents across the KCNH2 (or human Ether-a-go-go (hERG), Kᵥ11.1) and KCNQ 1 (or KᵥLQT1, Kᵥ7.1) channels, I_{Kr} and I_{Ks}, respectively, are reduced, which prolongs the cardiac repolarization phase. On a surface electrocardiogram (ECG), this delay is reflected by a prolonged QT interval predisposing patients to life-threatening arrhythmias. Current treatment options for LQTS patients include anti-arrhythmic drugs (including beta-blockers), left cardiac sympathetic denervation, and/or the implantation of a cardioverter-defibrillator (3).

Some LQTS patients enter periods of electrical storms and are resistant to standard therapy, and endure repeated defibrillation shocks and increased mortality (2). Additionally, patients with the type 2 form of LQTS respond less well to conventional treatment compared to LQTS1 individuals (10-15).

LQTS3 is caused by gain-of-function mutations in the SCN5A-encoded Nav1.5 sodium ion (Na⁺) channel.

### SUMMARY

In one aspect, described herein is an isolated monoclonal antibody or antigen-binding fragment thereof that specifically binds human KCNQ1, the antibody comprising a set of 6 CDRs set forth in SEQ ID NOs: 1-6.

In some embodiments, the antibody comprises a light chain variable region amino acid sequence set forth in SEQ ID NO: 7. In some embodiments, the antibody comprises a heavy chain variable region chain amino acid sequence set forth in SEQ ID NO: 8. In some embodiments, the antibody binds to an epitope comprising the amino acid sequence set forth in SEQ ID NO: 10. In some embodiments, the antibody is a murine antibody.

Antigen-binding fragments are also contemplated. In some embodiments, the antigen-binding fragment is a Fab fragment or an scFv.

Nucleic acids encoding an antibody described herein, as well as vectors and host cells comprising vectors encoding the nucleic acids are also contemplated.

In another aspect, described herein is a method of treating a subject suffering from long QT syndrome (LQTS) comprising administering the antibody to the subject in an amount effective to treat long QT syndrome. In some embodiments, the long QT syndrome is LQTS1, LQTS2 or LQTS3.

In some embodiments, the methods described herein optionally further comprise administering a standard of care to the subject for the treatment of long QT syndrome. In some embodiments, the standard of care is a beta-blocker, an implantable cardioverter-defibrillator (ICD), or a left cardiac sympathetic denervation.

In some embodiments, the subject is also suffering from cardiomyopathy, diabetes, epilepsy or neurological comorbidities.

In some embodiments, administering the antibody results in shorter cardiac repolarization compared to a subject that did not receive the antibody. In some embodiments, administering the antibody results in the reduced incidence of ventricular tachyarrhythmias including sudden cardiac arrest compared to a subject that did not receive the antibody. In some embodiments, administering the antibody does not affect KCNQ1 channel expression in the subject.

### BRIEF DESCRIPTION OF THE FIGURES

FIG. 1. Effect of 6 different monoclonal KCNQ1 antibodies on I_{Ks} recorded in Chinese Hamster Ovary (CHO) KCNQ1⁺/KCNE1⁺ cells. I_{Ks} step (FIG. 1A) and tail (FIG. 1B) current densities as a function of the test potential. Indicated are mean±SEM, comparing control cells (n=16, mean cell capacitance 17.2±2.2 pF) and cells treated with 30 μg/ml of the selected monoclonal antibodies: IgG2a 8-F11-D4 (n=15, mean cell capacitance 16.7±1.1 pF), IgG1 5-D4-D1 (n=12, mean cell capacitance 18.2±1.9 pF), IgG2b 7-D12-B11-D12 (n=8, mean cell capacitance 18.4±2.5 pF), IgG2b 9-F5-H2-2-G11-F6 (n=8, mean cell capacitance 20.8±3.4 pF), IgG2b 10-F10-D7-B1 (n=8, mean cell capacitance 17.7±1.9 pF), IgG1 3-A11-H3-F3 (n=5, mean cell capacitance 26.0±6.3 pF).

FIG. 2. Effect of IgG2a 8-F11-D4 monoclonal antibody on I_{Ks}. Representative I_{Ks} current traces recorded in CHO KCNQ1⁺/KCNE1⁺ cells under control condition (FIG. 2A, cell capacitance 13.72 pF) and in the presence of 30 μg/ml IgG2a 8-F11-D4 (FIG. 2B, 13.59 pF). (FIG. 2C) and (FIG. 2D) I_{Ks} and tail current densities as a function of the test potential. Indicated are mean±SEM, comparing control cells (n=16) and cells treated with 30 μg/ml IgG2a 8-F11-D4 (n=15). *P<0.05, **P<0.01, ***P<0.001, ****P<0.0001. (FIG. 2E) Voltage-dependent activation of I_{Ks}. In the presence of 30 μg/ml IgG2a 8-F11-D4, I_{Ks} currents were activated at more negative potentials compared with control cells, without manifest effect on the slope factor k (control: V_{1/2}=23.6±1.3 mV, slope factor k=16.9±1.2 mV; IgG2a 8-F11-D4: V_{1/2}=14.6±0.9 mV, slope factor k=17.5±1.0 mV). P<0.0001 when comparing V_{1/2} between control versus IgG2a 8-F11-D4; P value not significant when comparing slope factor k between control versus IgG2a 8-F11-D4. (FIG. 2F) Voltage-dependent deactivation of I_{Ks}. 30 μg/ml IgG2a 8-F11-D4 led to a leftward shift of the voltage-dependence of deactivation, while not affecting the slope factor k (control: V_{1/2}=20.0±1.4 mV, slope factor k=15.9±1.4 mV; IgG2a 8-F11-D4: V_{1/2}=8.7±1.1 mV, slope factor k=14.2±1.1 mV). P<0.0001 when comparing V_{1/2} between control versus IgG2a 8-F11-D4; P value not significant when comparing slope factor k between control versus IgG2a 8-F11-D4.

FIG. 3. Effect of IgG2a 8-F11-D4 monoclonal antibody on CHO KCNQ1⁺/KCNE1⁺ cells compared to anti-KCNQ1 polyclonal antibody. (FIG. 3A) and (FIG. 3B) I_{Ks} step and tail current densities as a function of the test potential. Indicated are mean±SEM, comparing control cells (n=16) and cells treated with 30 μg/ml IgG2a 8-F11-D4 (n=15) versus 30 μg/ml anti-KCNQ 1 polyclonal antibody (n=23). **P<0.01, ***P<0.001, ****P<0.0001 when comparing control versus 30 μg/ml IgG2a 8-F11-D4. P>0.05 when comparing cells treated with 30 μg/ml IgG2a 8-F11-D4 versus 30 μg/ml anti-KCNQ1 polyclonal antibody, except for I_{Ks} step current at +70 mV (P=0.01).

FIG. 4. Effect of IgG2a 8-F11-D4 monoclonal antibody on hiPSC-CMCs. Representative action potential traces recorded in hiPSC-CMCs under control condition and in the presence of 30 μg/ml IgG2a 8-F11-D4 (FIG. 4A). (FIG. 4B) The bars represent the mean±SEM of APD₉₀ of control cells (n=15) and cardiomyocytes treated with 5 μg/ml (n=4), 10 μg/ml (n=10), 20 μg/ml (n=11), 30 μg/ml (n=14) and 60 μg/ml (n=14) IgG2a 8-F11-D4. **P<0.01, ***P<0.001, ****P<0.0001. All measurements were performed at 37° C. (FIG. 4C) Concentration-response curve for the absolute APD₉₀ reduction effect at 5 different concentrations of IgG2a 8-F11-D4. Data are expressed as mean APD₉₀±SEM. The antibody concentration is plotted as a base 10 logarithmic scale. With a sigmoidal curve fit to the data, the half-maximal effective concentration (EC₅₀) was determined at 5.7 μg/ml (R square=0.9967). APD₉₀=action potential duration at 90% repolarization; hiPSC-CMC=human induced-pluripotent stem cell-derived cardiomyocyte.

FIG. 5. Effect of IgG2a 8-F11-D4 monoclonal antibody on hiPSC-CMCs in the context of pharmacological LQTS type 2. (FIG. 5A) Representative action potentials recorded in hiPSC-CMCs challenged with 10 nM E-4031 showing EADs, arrhythmic beating degenerating in beating arrest. (FIG. 5B) Representative action potentials recorded in hiPSC-CMCs treated with 30 μg/ml IgG2a 8-F11-D4 and challenged with 10 nM E-4031. (FIG. 5C) The bars represent the incidence of EADs, arrhythmic beating and beating arrest in hiPSC-CMCs challenged with 10 nM E-4031 (n=7)±30 μg/ml IgG2a 8-F11-D4 (n=8). (FIG. 5D) The bars represent the mean APD₉₀±SEM of hiPSC-CMCs challenged with 10 nM E-4031 (n=7)±30 μg/ml IgG2a 8-F11-D4 (n=8). ***P<0.001. APD₉₀=action potential duration at 90% repolarization; EAD=early afterdepolarization; hiPSC-CMC=human induced-pluripotent stem cell-derived cardiomyocyte.

FIG. 6. Effect of IgG2a 8-F11-D4 monoclonal antibody on hiPSC-CMCs in the context of pharmacological LQTS type 3. (FIG. 6A) Representative action potentials recorded in hiPSC-CMCs challenged with 5 nM ATX-II leading to EADs and arrhythmic beating. (FIG. 6B) Representative action potentials recorded in hiPSC-CMCs treated with 30 μg/ml IgG2a 8-F11-D4 and challenged with 5 nM ATX-II. (FIG. 6C) The bars represent the incidence of EADs and arrhythmic beating in hiPSC-CMCs challenged with 5 nM ATX-II (n=16)±30 μg/ml IgG2a 8-F11-D4 (n=14). (FIG. 6D) The bars represent the mean APD₉₀±SEM of hiPSC-CMCs challenged with 5 nM ATX-II (n=16)±30 μg/ml IgG2a 8-F11-D4 (n=14). ****P<0.0001. APD₉₀=action potential duration at 90% repolarization; EAD=early afterdepolarization; hiPSC-CMC=human induced-pluripotent stem cell-derived cardiomyocyte.

FIG. 7. Conformational epitope mapping of IgG2a 8-F11-D4. (FIG. 7A) PEPperCHIP® peptide microarray covering the entire sequence of KCNQ1 protein translated into overlapping constrained cyclic peptides of 7, 10 and 13 amino acid lengths. In total the microarray contained 2043 different peptides printed in duplicate, framed by additional HA peptides (YPYDVPDYAG, 134 spots) serving as control. The microarrays were probed with 0.1 μg/ml IgG2a 8-F11-D4 followed by staining with anti-mouse IgG (red) and anti-HA IgG (green). Sequences of reactive spots (red) are shown. Residues potentially contributing to antibody binding are highlighted in blue. (FIG. 7B) Intensity plots of the peptide microarray demonstrating clear epitope peaks for the consensus motif VEFG. Significantly weaker interactions were found for peptides with the consensus motifs FGTE and VDGY.

FIG. 8. Molecular interaction between KCNQ1 and IgG2a 8-F11-D4. (FIG. 8A) Predicted structure of the human KCNQ1 channel and murine IgG2a 8-F11-D4. The complementary-determining regions (CDRs) of the antibody are highlighted in orange. The target epitope on the third extracellular domain of KCNQ1 is colored in violet, while the transmembrane segments of the channel are illustrated in green. (FIG. 8B) Predicted binding sites of KCNQ1 and IgG2a. The table shows the amino acid residues of the light (VL) and heavy chains (VH) of the antibody involved in the hydrogen and ionic bonding to the KCNQ1 channel. The respective binding energies and distances are listed. Molecular graphics were rendered using Molecular Operating Environment software (MOE, Chemical Computing Group).

FIG. 9. Binding of IgG2a 8-F11-D4 to KCNQ1 channel peptide. Different concentrations of KCNQ1 channel peptide (serially diluted by two-fold) were injected for 120 s followed by 600 s of dissociation. Representative binding sensorgrams of injections performed in triplicate.

FIGS. 10A and 10B. Binding of IgG2a 8-F11-D4 to cell surface KCNQ1. Commercially available rabbit polyclonal KCNQ1 antibody (FIG. 10B) and the mouse monoclonal IgG2a 8-F11-D4 (FIG. 10A) antibody bind to KCNQ1 in two different CHO cell lines-one in which KCNQ1 and KCNE1 are fused, and whose

FIG. 11. IgG2a 8-F11-D4 shortens rabbit baseline QT interval. Telemetry-instrumented rabbits treated with varying doses of IgG2a 8-F11-D4 exhibit a shortening of the baseline QT interval in the rabbits at all tested doses. Shortening was observed between 6-12 h post-dose administration, with a steady-state level of shortening being observed at 12-20 h post-dose.

FIG. 12. IgG2a 8-F11-D4 protects against drug-induced QT prolongation and torsade de pointes. Rabbits treated with IgG2a 8-F11-D4 monoclonal antibody provided protection against drug-induced QT prolongation in a dose-dependent manner. 40 mg/kg protected against drug-induced torsades de pointes.

FIG. 13A-13B13. IgG2a 8-F11-D4 treatment increases current density in HEK293 cells expressing KCNQ1/KCNE1. (FIG. 13A) Current density was increased in the presence of IgG2a 8-F11-D4 monoclonal antibody at a concentration of 30 μg/mL. (FIG. 13B) Current density was increased in the presence of IgG2a 8-F11-D4 monoclonal antibody at a concentration of 60 μg/mL.

FIGS. 14A and 14B. IgG2a 8-F11-D4 increases the KCNQ1/KCNE1 step current density I_{Ks} step current densities were increased at membrane potentials more positive than −20 mV at 30 and 60 ug/mL IgG2a 8-F11-D4 monoclonal antibody (FIG. 14A) compared to the control (FIG. 14B).

FIGS. 15A and 15B. IgG2a 8-F11-D4 increases the KCNQ1/KCNE1 tail current density Tail current densities (FIG. 15B) were increased at membrane potentials more positive than −20 mV at 60 ug/mL IgG2a 8-F11-D4 monoclonal antibody (FIG. 15a) compared to the control (FIG. 15B).

FIG. 16. Kinetic measurements of mAb binding to 20aa KCNQ1 target sequence. (FIG. 16A) Representative Octet sensorgrams for mAb binding (at 100 mM) to 1 mM N-terminally biotinylated KCNQ1 peptide (Nterm-Biotin-(CH2O)4-AEKDAVNESGRVEFGSYADA-Cterm (SEQ ID NO: 10) in 1× PBS pH 7.4, 1× kinetic buffer and 1% BSA. (FIG. 16B) mAb-KCNQ1 peptide interactions were analyzed by 1:1 binding model and the reported KDs were derived from the antibody dissociation (koff) and association (kon) rate constants. A table with mean KD values were calculated based on duplicate runs.

### DETAILED DESCRIPTION

The present disclosure is based on the discovery that anti-Potassium Voltage-Gated Channel Subfamily Q Member 1 (KCNQ1) monoclonal antibodies act as agonists on the I_{Ks} channel. As shown in the Examples, doubling the KCNQ1 antibody concentration increased the I_{Ks} current density in a concentration-dependent manner in CHO KCNQ1⁺/KCNE1⁺ cells. Patch clamp recordings disclosed a dual effect of KCNQ1 antibodies: a negative shift in voltage-dependence of activation and a marked slowing of I_{Ks} deactivation. Taken together, the data provided herein demonstrates the therapeutic potential of KCNQ1 monoclonal antibodies by enhancing repolarization reserve and restoring electrical stability in LQTS2.

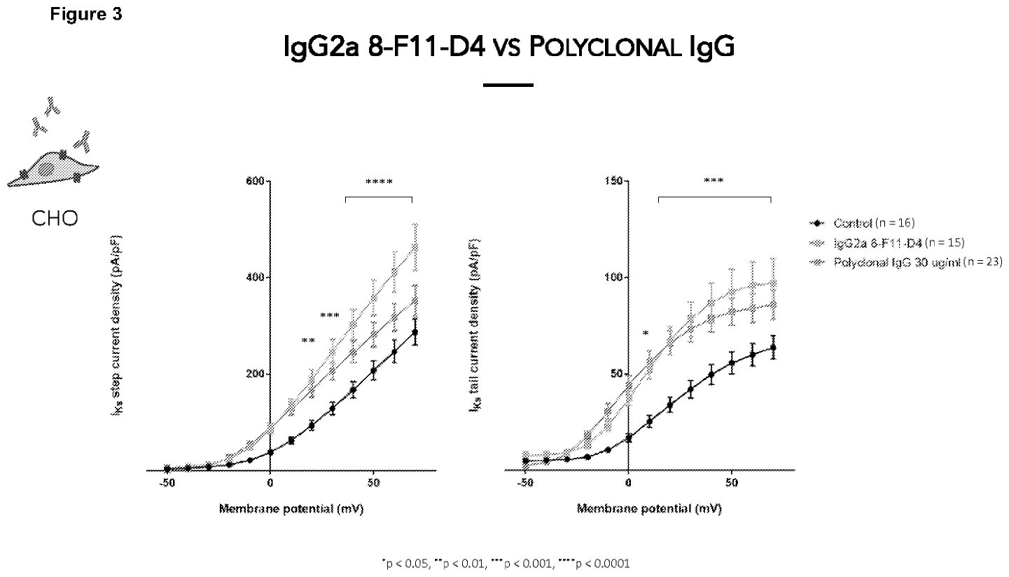

A previous study (Maguy et al. JACC, 75:2140-2152, 2020 the disclosure of which is incorporated by reference in its entirety) has shown the effects of a polyclonal antibody against KCNQ1. Described herein is a monoclonal antibody that specifically binds KCNQ1 that is as effective at increasing I_{Ks} currents under voltage-clamp conditions as anti-KCNQ1 polyclonal antibodies. As shown in FIG. 3, anti-KCNQ1 monoclonal antibody increased I_{Ks} currents similarly to the anti-KCNQ1 polyclonal antibody previously disclosed in Maguy et al. (supra).

### Antibodies

In one aspect, described herein is an a monoclonal antibody that specifically binds human KCNQ1 (SEQ ID NO: 9). The antibody may be any type of antibody, i.e., immunoglobulin, known in the art. In exemplary embodiments, the antibody is an antibody of class or isotype IgA, IgD, IgE, IgG, or IgM. In exemplary embodiments, the antibody described herein comprises one or more alpha, delta, epsilon, gamma, and/or mu heavy chains. In exemplary embodiments, the antibody described herein comprises one or more kappa or lambda light chains.

The term “specifically binds” as used herein means that the antibody (or antigen binding fragment) preferentially binds an antigen (e.g., KCNQ1) over other proteins. In some embodiments, “specifically binds” means the antibody has a higher affinity for the antigen than for other proteins. Antibodies that specifically bind an antigen may have a binding affinity for the antigen of less than or equal to 1×10⁻⁷ M, less than or equal to 2×10⁻⁷ M, less than or equal to 3×10⁻⁷ M, less than or equal to 4×10⁻⁷ M, less than or equal to 5×10⁻⁷ M, less than or equal to 6×10⁻⁷ M, less than or equal to 7×10⁻⁷ M, less than or equal to 8×10⁻⁷ M, less than or equal to 9×10⁻⁷ M, less than or equal to 1×10⁻⁸ M, less than or equal to 2×10⁻⁸ M, less than or equal to 3×10⁻⁸ M, less than or equal to 4×10⁻⁸ M, less than or equal to 5×10⁻⁸ M, less than or equal to 6×10⁻⁸ M, less than or equal to 7×10⁻⁸ M, less than or equal to 8×10⁻⁸ M, less than or equal to 9×10⁻⁸ M, less than or equal to 1×10⁻⁹ M, less than or equal to 2×10⁻⁹ M, less than or equal to 3×10⁻⁹ M, less than or equal to 4×10⁻⁹ M, less than or equal to 5×10⁻⁹ M, less than or equal to 6×10⁻⁹ M, less than or equal to 7×10⁻⁹ M, less than or equal to 8×10⁻⁹ M, less than or equal to 9×10⁻⁹ M, less than or equal to 1×10⁻¹⁰ M, less than or equal to 2×10⁻¹⁰ M, less than or equal to 3×10⁻¹⁰ M, less than or equal to 4×10⁻¹⁰ M, less than or equal to 5×10⁻¹⁰ M, less than or equal to 6×10⁻¹⁰ M, less than or equal to 7×10⁻¹⁰ M, less than or equal to 8×10⁻¹⁰ M, less than or equal to 9×10⁻¹⁰ M, less than or equal to 1×10⁻¹¹ M, less than or equal to 2×10⁻¹¹ M, less than or equal to 3×10⁻¹¹ M, less than or equal to 4×10⁻¹¹ M, less than or equal to 5×10⁻¹¹ M, less than or equal to 6×10⁻¹¹ M, less than or equal to 7×10⁻¹¹ M, less than or equal to 8×10⁻¹¹ M, less than or equal to 9×10⁻¹¹ M, less than or equal to 1×10⁻¹² M, less than or equal to 2×10⁻¹² M, less than or equal to 3×10⁻¹² M, less than or equal to 4×10⁻¹² M, less than or equal to 5×10⁻¹² M, less than or equal to 6×10⁻¹² M, less than or equal to 7×10⁻¹² M, less than or equal to 8×10⁻¹² M, or less than or equal to 9×10⁻¹² M, It will be appreciated that ranges having the values above as end points is contemplated in the context of the disclosure. For example, the antibody or antigen binding fragment thereof may bind KCNQ1 of SEQ ID NO: 9 with an affinity of about 1×10⁻⁷ M to about 9×10⁻¹² M or an affinity of 1×10⁻⁹ to about 9×10⁻¹².

In some or any embodiments, the antibody (or antigen binding fragment) binds to KCNQ1 of SEQ ID NO: 9, or a naturally occurring variant thereof, with an affinity (Kd) of less than or equal to 1×10⁻⁷ M, less than or equal to 1×10⁻⁸ M, less than or equal to 1×10⁻⁹ M, less than or equal to 1×10⁻¹⁰ M, less than or equal to 1×10⁻¹¹ M. or less than or equal to 1×10⁻¹² M. or ranging from 1×10⁻⁹ to 1×10⁻¹⁰, or ranging from 1×10⁻¹² to about 1×10⁻¹³. Affinity is determined using a variety techniques, examples of which include an affinity ELISA assay and a surface plasmon resonance (BIACORE™) assay.

In some embodiments, the antibody (or antigen binding fragment) binds to an epitope of KCNQ1 comprising amino acids 291-297 of SEQ ID NO: 9. In some embodiments, the antibody (or antigen binding fragment) binds to an epitope of KCNQ1 comprising amino acids 292-298 of SEQ ID NO: 9. In some embodiments, the antibody (or antigen binding fragment) binds to an epitope of KCNQ1 comprising amino acids 293-299 of SEQ ID NO: 9. In some embodiments, the antibody (or antigen binding fragment) binds to an epitope of KCNQ1 comprising amino acids 294-300 of SEQ ID NO: 9. In some embodiments, the antibody (or antigen binding fragment) binds to an epitope of KCNQ1 comprising amino acids 288-297 of SEQ ID NO: 9. In some embodiments, the antibody (or antigen binding fragment) binds to an epitope of KCNQ1 comprising amino acids 289-298 of SEQ ID NO: 9. In some embodiments, the antibody (or antigen binding fragment) binds to an epitope of KCNQ1 comprising amino acids 290-299 of SEQ ID NO: 9. In some embodiments, the antibody (or antigen binding fragment) binds to an epitope of KCNQ1 comprising amino acids 291-300 of SEQ ID NO: 9. In some embodiments, the antibody (or antigen binding fragment) binds to an epitope of KCNQ1 comprising amino acids 292-301 of SEQ ID NO: 9. In some embodiments, the antibody (or antigen binding fragment) binds to an epitope of KCNQ1 comprising amino acids 293-302 of SEQ ID NO: 9. In some embodiments, the antibody (or antigen binding fragment) binds to an epitope of KCNQ1 comprising amino acids 294-303 of SEQ ID NO: 9. In some embodiments, the antibody (or antigen binding fragment) binds to an epitope of KCNQ1 comprising amino acids 285-297 of SEQ ID NO: 9. In some embodiments, the antibody (or antigen binding fragment) binds to an epitope of KCNQ1 comprising amino acids 286-298 of SEQ ID NO: 9. In some embodiments, the antibody (or antigen binding fragment) binds to an epitope of KCNQ1 comprising amino acids 287-299 of SEQ ID NO: 9. In some embodiments, the antibody (or antigen binding fragment) binds to an epitope of KCNQ1 comprising amino acids 288-300 of SEQ ID NO: 9. In some embodiments, the antibody (or antigen binding fragment) binds to an epitope of KCNQ1 comprising amino acids 289-301 of SEQ ID NO: 9. In some embodiments, the antibody (or antigen binding fragment) binds to an epitope of KCNQ1 comprising amino acids 290-302 of SEQ ID NO: 9. In some embodiments, the antibody (or antigen binding fragment) binds to an epitope of KCNQ1 comprising amino acids 291-303 of SEQ ID NO: 9. In some embodiments, the antibody (or antigen binding fragment) binds to an epitope of KCNQ1 comprising amino acids 292-304 of SEQ ID NO: 9. In some embodiments, the antibody (or antigen binding fragment) binds to an epitope of KCNQ1 comprising amino acids 293-305 of SEQ ID NO: 9. In some embodiments, the antibody (or antigen binding fragment) binds to an epitope of KCNQ1 comprising amino acids 294-306 of SEQ ID NO: 9.

In some embodiments, the antibody (or antigen binding fragment) cross-blocks or is cross-blocked by an antibody that binds to an epitope of KCNQ1 comprising amino acids 291-297 of SEQ ID NO: 9. The terms “cross-block,” “cross-blocked,” and “cross-blocking” are used interchangeably herein to mean the ability of an antibody to interfere with the binding of other antibodies to KCNQ1. The extent to which an antibody is able to interfere with the binding of another to KCNQ1 and therefore whether it can be said to cross-block, can be determined using competition binding assays. In some aspects, a cross-blocking antibody or fragment thereof reduces KCNQ1 binding of a reference antibody between about 40% and about 100%, such as about 60% and about 100%, specifically between 70% and 100%, and more specifically between 80% and 100%. A particularly suitable quantitative assay for detecting cross-blocking uses a BIACORE™ machine which measures the extent of interactions using surface plasmon resonance technology. Another suitable quantitative cross-blocking assay uses an ELISA-based approach to measure competition between antibodies in terms of their binding to KCNQ1.

In some embodiments, the antibody (or antigen binding fragment) cross-blocks or is cross-blocked by an antibody that binds to an epitope of KCNQ1 comprising amino acids 292-298 of SEQ ID NO: 9. In some embodiments, the antibody (or antigen binding fragment) cross-blocks or is cross-blocked by an antibody that binds to an epitope of KCNQ1 comprising amino acids 293-299 of SEQ ID NO: 9. In some embodiments, the antibody (or antigen binding fragment) cross-blocks or is cross-blocked by an antibody that binds to an epitope of KCNQ1 comprising amino acids 294-300 of SEQ ID NO: 9. In some embodiments, the antibody (or antigen binding fragment) cross-blocks or is cross-blocked by an antibody that binds to an epitope of KCNQ1 comprising amino acids 288-297 of SEQ ID NO: 9. In some embodiments, the antibody (or antigen binding fragment) cross-blocks or is cross-blocked by an antibody that binds to an epitope of KCNQ1 comprising amino acids 289-298 of SEQ ID NO: 9. In some embodiments, the antibody (or antigen binding fragment) cross-blocks or is cross-blocked by an antibody that binds to an epitope of KCNQ1 comprising amino acids 290-299 of SEQ ID NO: 9. In some embodiments, the antibody (or antigen binding fragment) cross-blocks or is cross-blocked by an antibody that binds to an epitope of KCNQ1 comprising amino acids 291-300 of SEQ ID NO: 9. In some embodiments, the antibody (or antigen binding fragment) binds to an epitope of KCNQ1 comprising amino acids 292-301 of SEQ ID NO: 9. In some embodiments, the antibody (or antigen binding fragment) cross-blocks or is cross-blocked by an antibody that binds to an epitope of KCNQ1 comprising amino acids 293-302 of SEQ ID NO: 9. In some embodiments, the antibody (or antigen binding fragment) cross-blocks or is cross-blocked by an antibody that binds to an epitope of KCNQ1 comprising amino acids 294-303 of SEQ ID NO: 9. In some embodiments, the antibody (or antigen binding fragment) cross-blocks or is cross-blocked by an antibody that binds to an epitope of KCNQ1 comprising amino acids 285-297 of SEQ ID NO: 9. In some embodiments, the antibody (or antigen binding fragment) cross-blocks or is cross-blocked by an antibody that binds to an epitope of KCNQ1 comprising amino acids 286-298 of SEQ ID NO: 9. In some embodiments, the antibody (or antigen binding fragment) cross-blocks or is cross-blocked by an antibody that binds to an epitope of KCNQ1 comprising amino acids 287-299 of SEQ ID NO: 9. In some embodiments, the antibody (or antigen binding fragment) cross-blocks or is cross-blocked by an antibody that binds to an epitope of KCNQ1 comprising amino acids 288-300 of SEQ ID NO: 9. In some embodiments, the antibody (or antigen binding fragment) cross-blocks or is cross-blocked by an antibody that binds to an epitope of KCNQ1 comprising amino acids 289-301 of SEQ ID NO: 9. In some embodiments, the antibody (or antigen binding fragment) cross-blocks or is cross-blocked by an antibody that binds to an epitope of KCNQ1 comprising amino acids 290-302 of SEQ ID NO: 9. In some embodiments, the antibody (or antigen binding fragment) cross-blocks or is cross-blocked by an antibody that binds to an epitope of KCNQ1 comprising amino acids 291-303 of SEQ ID NO: 9. In some embodiments, the antibody (or antigen binding fragment) cross-blocks or is cross-blocked by an antibody that binds to an epitope of KCNQ1 comprising amino acids 292-304 of SEQ ID NO: 9. In some embodiments, the antibody (or antigen binding fragment) cross-blocks or is cross-blocked by an antibody that binds to an epitope of KCNQ1 comprising amino acids 293-305 of SEQ ID NO: 9. In some embodiments, the antibody (or antigen binding fragment) cross-blocks or is cross-blocked by an antibody that binds to an epitope of KCNQ1 comprising amino acids 294-306 of SEQ ID NO: 9.

“CDR” refers to the complementarity determining region within antibody variable sequences. There are three CDRs in each of the variable regions of the heavy chain and the light chain, which are designated CDR1, CDR2 and CDR3, for each of the variable regions. The term “set of six CDRs” as used herein refers to a group of three CDRs that occur in the light chain variable region and heavy chain variable region, which are capable of binding the antigen. The exact boundaries of CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):73245 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

CDRs are obtained by, e.g., constructing polynucleotides that encode the CDR of interest and expression in a suitable host cell. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)).

In various aspects, the antibody (or antigen binding fragment thereof) comprises at least one CDR sequence having at least 75% identity (e.g., at least 75%, 80%, 85%, 90%, 95% or 100% identity) to a CDR selected from CDR-H1, CDR-H2, CDR-H3, CDR-L1, CDR-L2, and CDR-L3 wherein CDR-H1 has the sequence given in SEQ ID NO: 1, CDR-H2 has the sequence given in SEQ ID NO: 2, CDR-H3 has the sequence given in SEQ ID NO: 3, CDR-L1 has the sequence given in SEQ ID NO: 4, CDR-L2 has the amino acid sequence of “WAS” and CDR-L3 has the sequence given in SEQ ID NO: 6. In various aspects, the antibody (or antigen binding fragment thereof) comprises a CDR-H1 having the sequence given in SEQ ID NO: 1 with 3, 2, or 1 amino acid substitutions therein, CDR-H2 having the sequence given in SEQ ID NO: 2 with 3, 2, or 1 amino acid substitutions therein, CDR-H3 having the sequence given in SEQ ID NO: 3 with 3, 2, or 1 amino acid substitutions therein, CDR-L1 having the sequence given in SEQ ID NO: 4 with 3, 2, or 1 amino acid substitutions therein, CDR-L2 having the amino acid sequence of “WAS” with 3, 2, or 1 amino acid substitutions therein and CDR-L3 having the sequence given in SEQ ID NO: 6 with 3, 2, or 1 amino acid substitutions therein. The anti-KCNQ1 antibody, in various aspects, comprises two of the CDRs, three of the CDRs, four of the CDRs, five of the CDRs or all six of the CDRs. In a preferred embodiment, the anti-KCNQ1 antibody comprises a set of six CDRs as follows: CDR-H1 of SEQ ID NO: 1, CDR-H2 of SEQ ID NO: 2, CDR-H3 of SEQ ID NO: 3, CDR-L1 of SEQ ID NO: 4, CDR-L3 of SEQ ID NO: 6, and the CDR-L2 having the amino acid sequence “WAS.”

In some or any embodiments, the antibody comprises a light chain variable region comprising an amino acid sequence having at least 75% identity (e.g., at least 75%, 80%, 85%, 90%, 95% or 100% identity) to the amino acid sequence set forth in SEQ ID NO: 7 and/or a heavy chain variable region comprising an amino acid sequence having at least 75% identity (e.g., at least 75%, 80%, 85%, 90%, 95% or 100% identity) to the amino acid sequence set forth in SEQ ID NO: 8. In various aspects, the difference in the sequence compared to SEQ ID NO: 7 (or SEQ ID NO: 8) lies outside the CDR region in the corresponding sequences.

In some or any embodiments, the antibody (or antigen binding fragment) comprises a heavy chain variable region comprising an amino acid sequence set forth in SEQ ID NO: 8 and a light chain variable region comprising an amino acid sequence set forth in SEQ ID NO: 7.

Antigen binding fragments of the anti-KCNQ1 antibodies described herein are also contemplated. The antigen binding fragment can be any part of an antibody that has at least one antigen binding site, and the antigen binding fragment may be part of a larger structure (an “antibody product”) that retains the ability of the antigen binding fragment to recognize KCNQ1. For ease of reference, these antibody products that include antigen binding fragments are included in the disclosure herein of “antigen binding fragment.” Examples of antigen binding fragments, include, but are not limited to, Fab, F(ab′)₂, a monospecific or bispecific Fab₂, a trispecific Fab₃, scFv, dsFv, scFv-Fc, bispecific diabodies, trispecific triabodies, minibodies, a fragment of IgNAR (e.g., V-NAR), a fragment of hcIgG (e.g., VhH), bis-scFvs, fragments expressed by a Fab expression library, and the like. In exemplary aspects, the antigen binding fragment is a domain antibody, VhH domain, V-NAR domain, VH domain, VL domain, or the like. Antibody fragments of the disclosure, however, are not limited to these exemplary types of antibody fragments. In exemplary aspects, antigen binding fragment is a Fab fragment. In exemplary aspects, the antigen binding fragment comprises two Fab fragments. In exemplary aspects, the antigen binding fragment comprises two Fab fragments connected via a linker. In exemplary aspects, the antigen binding fragment comprises or is a minibody comprising two Fab fragments. In exemplary aspects, the antigen binding fragment comprises, or is, a minibody comprising two Fab fragments joined via a linker. Minibodies are known in the art. See, e.g., Hu et al., Cancer Res 56: 3055-3061 (1996). In exemplary aspects, the antigen binding fragment comprises or is a minibody comprising two Fab fragments joined via a linker, optionally, comprising an alkaline phosphatase domain.

A domain antibody comprises a functional binding unit of an antibody, and can correspond to the variable regions of either the heavy (V_{H}) or light (V_{L}) chains of antibodies. A domain antibody can have a molecular weight of approximately 13 kDa, or approximately one-tenth of a full antibody. Domain antibodies may be derived from full antibodies such as those described herein.

### Methods of Antibody or Antigen Binding Fragment Production

Suitable methods of making antibodies are known in the art. For instance, standard hybridoma methods are described in, e.g., Harlow and Lane (eds.), Antibodies: A Laboratory Manual, CSH Press (1988), and CA. Janeway et al. (eds.), Immunobiology, 5th Ed., Garland Publishing, New York, NY (2001)). Monoclonal antibodies for use in the methods of the disclosure may be prepared using any technique which provides for the production of antibody molecules by continuous cell lines in culture. These include but are not limited to the hybridoma technique originally described by Koehler and Milstein (Nature 256: 495-497, 1975), the human B-cell hybridoma technique (Kosbor et al., Immunol Today 4:72, 1983; Cote et al., Proc Natl Acad Sci 80: 2026-2030, 1983) and the EBV-hybridoma technique (Cole et al., Monoclonal Antibodies and Cancer Therapy, Alan R Liss Inc, New York N.Y., pp 77-96, (1985). Alternatively, other methods, such as EBV-hybridoma methods (Haskard and Archer, J. Immunol. Methods, 74(2), 361-67 (1984), and Roder et al., Methods Enzymol., 121, 140-67 (1986)), and bacteriophage vector expression systems (see, e.g., Huse et al., Science, 246, 1275-81 (1989)) are known in the art. Further, methods of producing antibodies in non-human animals are described in, e.g., U.S. Pat. Nos. 5,545,806, 5,569,825, and 5,714,352, and U.S. Patent Application Publication No. 2002/0197266 A1). Antibodies may also be produced by inducing in vivo production in the lymphocyte population or by screening recombinant immunoglobulin libraries or panels of highly specific binding reagents as disclosed in Orlandi et al (Proc Natl Acad Sci 86: 3833-3837; 1989), and Winter G and Milstein C (Nature 349: 293-299, 1991). If the full sequence of the antibody or antigen-binding fragment is known, then methods of producing recombinant proteins may be employed. See, e.g., “Protein production and purification” Nat Methods 5(2): 135-146 (2008). In some embodiments, the antibodies (or antigen binding fragments) are isolated from cell culture or a biological sample if generated in vivo.

Phage display also can be used to generate the antibody of the present disclosures. In this regard, phage libraries encoding antigen-binding variable (V) domains of antibodies can be generated using standard molecular biology and recombinant DNA techniques (see, e.g., Sambrook et al. (eds.), Molecular Cloning, A Laboratory Manual, 3rd Edition, Cold Spring Harbor Laboratory Press, New York (2001)). Phage encoding a variable region with the desired specificity are selected for specific binding to the desired antigen, and a complete or partial antibody is reconstituted comprising the selected variable domain. Nucleic acid sequences encoding the reconstituted antibody are introduced into a suitable cell line, such as a myeloma cell used for hybridoma production, such that antibodies having the characteristics of monoclonal antibodies are secreted by the cell (see, e.g., Janeway et al., supra, Huse et al., supra, and U.S. Pat. No. 6,265,150). Related methods also are described in U.S. Pat. Nos. 5,403,484; 5,571,698; 5,837,500; 5,702,892. The techniques described in U.S. Pat. Nos. 5,780,279; 5,821,047; 5,824,520; 5,855,885; 5,858,657; 5,871,907; 5,969,108; 6,057,098; and 6,225,447.

Antibodies can be produced by transgenic mice that are transgenic for specific heavy and light chain immunoglobulin genes. Such methods are known in the art and described in, for example U.S. Pat. Nos. 5,545,806 and 5,569,825, and Janeway et al., supra.

Compositions comprising one, two, and/or three CDRs of a heavy chain variable region or a light chain variable region of a monoclonal antibody can be generated. The CDRs of exemplary antibodies are provided herein as SEQ ID NOs: 1-6. Techniques for cloning and expressing nucleotide and polypeptide sequences are well-established in the art (see, e.g., Sambrook et al., Molecular Cloning: A Laboratory Manual, 2nd Edition, Cold Spring Harbor, New York (1989)). The amplified CDR sequences are ligated into an appropriate expression vector. The vector comprising one, two, three, four, five and/or six cloned CDRs optionally contains additional polypeptide encoding regions linked to the CDR.

Chemically constructed bispecific antibodies may be prepared by chemically cross-linking heterologous Fab or F(ab′)₂ fragments by means of chemicals such as heterobifunctional reagent succinimidyl-3-(2-pyridyldithiol)-propionate (SPDP, Pierce Chemicals, Rockford, Ill.). The Fab and F(ab′)₂ fragments can be obtained from intact antibody by digesting it with papain or pepsin, respectively (Karpovsky et al., J. Exp. Med. 160:1686-701(1984); Titus et al., J. Immunol., 138:4018-22 (1987)).

Methods of testing antibodies for the ability to bind to an epitope of KCNQ1, regardless of how the antibodies are produced, are known in the art and include, e.g., radioimmunoassay (RIA), ELISA, Western blot, immunoprecipitation, surface plasmon resonance (e.g., Biacore), and competitive inhibition assays (see, e.g., Janeway et al., infra, and U.S. Patent Application Publication No. 2002/0197266).

Antibody fragments that contain the antigen binding, or idiotype, of the antibody molecule may be generated by techniques known in the art. For example, a F(ab′)₂ fragment may be produced by pepsin digestion of the antibody molecule; Fab′ fragments may be generated by reducing the disulfide bridges of the F(ab′)₂ fragment; and two Fab′ fragments which may be generated by treating the antibody molecule with papain and a reducing agent. The disclosure is not limited to enzymatic methods of generating antigen binding fragments; the antigen binding fragment may be a recombinant antigen binding fragment produced by expressing a polynucleotide encoding the fragment in a suitable host cell.

The heavy chains of the monoclonal antibodies described herein may further comprise one or more mutations that affect binding of the antibody containing the heavy chains to one or more Fc receptors. One of the functions of the Fc portion of an antibody is to communicate to the immune system when the antibody binds its target. This is commonly referenced as “effector function.” Communication leads to antibody-dependent cellular cytotoxicity (ADCC), antibody-dependent cellular phagocytosis (ADCP), and/or complement dependent cytotoxicity (CDC). ADCC and ADCP are mediated through the binding of the Fc to Fc receptors on the surface of cells of the immune system. CDC is mediated through the binding of the Fc with proteins of the complement system, e.g., C1q.

The effector function of an antibody can be increased, or decreased, by introducing one or more mutations into the Fc. Embodiments of the invention include heterodimeric antibodies, having an Fc engineered to increase effector function (U.S. Pat. No. 7,317,091 and Strohl, Curr. Opin. Biotech., 20:685-691, 2009; both incorporated herein by reference in its entirety).

In various embodiments, the disclosure provides a nucleic acid comprising a nucleotide sequence that encodes the heavy chain variable region and/or light chain variable region of an antibody as described herein.

Also contemplated is a vector comprising the nucleic acid encoding the antibody. In various embodiments, the nucleic acid encoding a heavy chain variable region and light chain variable region are expressed on the same vector or different vectors.

Further provided is a host cell comprising a nucleic acid encoding an antibody heavy and/or light chain variable region or vector expressing said nucleic acid. In some embodiments, the host cell is an eukaryotic cell.

A single-chain variable region fragments (scFv), which consists of a truncated Fab fragment comprising the variable (V) domain of an antibody heavy chain linked to a V domain of an antibody light chain via a synthetic peptide, can be generated using routine recombinant DNA technology techniques (see, e.g., Janeway et al., supra). Similarly, disulfide-stabilized variable region fragments (dsFv) can be prepared by recombinant DNA technology (see, e.g., Reiter et al., Protein Engineering, 7, 697-704 (1994)).

Recombinant antibody fragments, e.g., scFvs, can also be engineered to assemble into stable multimeric oligomers of high binding avidity and specificity to different target antigens. Such diabodies (dimers), triabodies (trimers) or tetrabodies (tetramers) are well known in the art, see e.g., Kortt et al., Biomol Eng. 2001 18:95-108, (2001) and Todorovska et al., J Immunol Methods. 248:47-66, (2001).

### Detection Methods

It is sometimes desirable to detect the presence or measure the amount of KCNQ1 in a sample. In this regard, the disclosure provides a method of using the antibody or fragment thereof described herein to measure the amount of KCNQ1 in a sample. To determine a measurement of KCNQ1, a biological sample from a mammalian subject is contacted with an anti-KCNQ1 antibody (or antigen binding fragment thereof) described herein for a time sufficient to allow immunocomplexes to form. Immunocomplexes formed between the antibody and KCNQ1 in the sample are then detected. The amount of KCNQ1 in the biological sample is optionally quantitated by measuring the amount of the immunocomplex formed between the antibody and the KCNQ1. For example, the antibody can be quantitatively measured if it has a detectable label, or a secondary antibody can be used to quantify the immunocomplex.

In some embodiments, the biological sample comprises a tissue sample, a cell sample, or a biological fluid sample, such as blood, saliva, serum, or plasma.

Conditions for incubating an antibody with a test sample vary. Incubation conditions depend on the format employed in the assay, the detection methods employed, and the type and nature of the antibody used in the assay. One skilled in the art will recognize that any one of the commonly available immunological assay formats can readily be adapted to employ the antibodies (or fragments thereof) of the present disclosure. Examples of such assays can be found in Chard, T., An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, FL Vol. 1 (1982), Vol. 2 (1983), Vol. 3 (1985); Tijssen, P., Practice and Theory of immunoassays: Laboratory Techniques in Biochemistry and Molecular Biology, Elsevier Science Publishers, Amsterdam, The Netherlands (1985). The test sample used in the above-described method will vary based on the assay format, nature of the detection method and the tissues, cells or fluids used as the sample to be assayed.

The assay described herein may be useful in, e.g., evaluating the efficacy of a particular therapeutic treatment regimen in animal studies, in clinical trials, or in monitoring the treatment of an individual patient.

In some embodiments, anti-KCNQ1 antibody (or antigen binding fragment thereof) is attached to a solid support, and binding is detected by detecting a complex between the KCNQ1 and the antibody (or antigen binding fragment thereof) on the solid support. The antibody (or fragment thereof) optionally comprises a detectable label and binding is detected by detecting the label in the KCNQ1-antibody complex.

Detection of the presence or absence of a KCNQ1-antibody complex can be achieved by using any method known in the art. For example, the transcript resulting from a reporter gene transcription assay of a KCNQ1 peptide interacting with a target molecule (e.g., antibody) typically encodes a directly or indirectly detectable product (e.g., β-galactosidase activity and luciferase activity). For cell free binding assays, one of the components usually includes, or is coupled to, a detectable label. A wide variety of labels can be used, such as those that provide direct detection (such as radioactivity, luminescence, optical or electron density) or indirect detection (such as epitope tag such as the FLAG epitope, enzyme tag such as horseradish peroxidase). The label can be bound to the antibody, or incorporated into the structure of the antibody.

A variety of methods can be used to detect the label, depending on the nature of the label and other assay components. For example, the label can be detected while bound to the solid substrate or subsequent to separation from the solid substrate. Labels can be directly detected through optical or electron density, radioactive emissions, nonradiative energy transfers or indirectly detected with antibody conjugates, or streptavidin-biotin conjugates. Methods for detecting the labels are well known in the art.

### Pharmaceutical Compositions

Pharmaceutical compositions comprising an anti-KCNQ1 antibody or antigen binding fragment thereof described herein are also contemplated. In some embodiments, the pharmaceutical composition contains formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine, proline, methionine or lysine); antimicrobials; antioxidants (such as reducing agents, oxygen/free-radical scavengers, and chelating agents (e.g., ascorbic acid, EDTA, sodium sulfite or sodium hydrogen-sulfite)); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter-ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, REMINGTON'S PHARMACEUTICAL SCIENCES, 18^{th} Edition, (A. R. Genrmo, ed.), 1990, Mack Publishing Company.

Selection of the particular formulation materials described herein may be driven by, for example, the intended route of administration, delivery format and desired dosage. See, for example, REMINGTON'S PHARMACEUTICAL SCIENCES, supra. The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. In specific embodiments, pharmaceutical compositions comprise Tris buffer of about pH 7.0-8.5, or acetate buffer of about pH 4.0-5.5, and may further include sorbitol or a suitable substitute therefor. In certain embodiments, the composition may be prepared for storage by mixing the selected composition having the desired degree of purity with optional formulation agents (REMINGTON'S PHARMACEUTICAL SCIENCES, supra) in the form of a lyophilized cake or an aqueous solution. Further, in some embodiments, the antibody or (antigen binding fragment thereof) may be formulated as a lyophilizate using appropriate excipients such as sucrose.

The pharmaceutical compositions of the invention can be selected for parenteral delivery. Alternatively, the compositions may be selected for inhalation or for delivery through the digestive tract, such as orally. Preparation of such pharmaceutically acceptable compositions is within the skill of the art. The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the composition may be provided in the form of a pyrogen-free, parenterally acceptable aqueous solution comprising the desired antibody or fragment in a pharmaceutically acceptable vehicle. A particularly suitable vehicle for parenteral injection is sterile distilled water in which the antibody or fragment is formulated as a sterile, isotonic solution, properly preserved. In certain embodiments, implantable drug delivery devices may be used to introduce the desired antibody (or antigen binding fragment thereof).

Additional pharmaceutical compositions, including formulations involving antigen binding proteins in sustained- or controlled-delivery formulations are contemplated herein. Techniques for formulating a variety of other sustained- or controlled-delivery means, such as liposome carriers, bio-erodible microparticles or porous beads and depot injections, are available in the art. See, for example, International Patent Application No. PCT/US93/00829, which is incorporated by reference and describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP058481, each of which is incorporated by reference), copolymers of L-glutamic acid and gamma ethyl-L-glutamate (Sidman et al., 1983, Biopolymers 2:547-556), poly (2-hydroxyethyl-methacrylate) (Langer et al., 1981, J. Biomed. Mater. Res. 15:167-277 and Langer, 1982, Chem. Tech. 12:98-105), ethylene vinyl acetate (Langer et al., 1981, supra) or poly-D(-)-3-hydroxybutyric acid (European Patent Application Publication No. EP133988). Sustained release compositions may also include liposomes that can be prepared by any of several methods known in the art. See, e.g., Eppstein et al., 1985, Proc. Natl. Acad. Sci. U.S.A. 82:3688-3692; European Patent Application Publication Nos. EP036676; EP088046 and EP143949, incorporated by reference.

Embodiments of the antibody formulations can further comprise one or more preservatives.

Administration of the compositions described herein will be via any common route so long as the target tissue is available via that route. The pharmaceutical compositions may be introduced into the subject by any conventional method, e.g., by intravenous, subcutaneous, intradermal, intramuscular, intramammary, intraperitoneal, intrathecal, intraocular, retrobulbar, intrapulmonary (e.g., term release); by oral, sublingual, nasal, anal, vaginal, or transdermal delivery, or by surgical implantation at a particular site.

### Dosage

In some embodiments, one or more doses of the antibody or antigen binding fragment are administered in an amount and for a time effective to treat a long QT syndrome (LQTS) subject. For example, one or more administrations of an antibody or antigen binding fragment thereof described herein are optionally carried out over a therapeutic period of, for example, about 1 week to about 24 months (e.g., about 1 month to about 12 months, about 1 month to about 18 months, about 1 month to about 9 months or about 1 month to about 6 months or about 1 month to about 3 months). In some embodiments, a subject is administered one or more doses of an antibody or fragment thereof described herein over a therapeutic period of, for example about 1 month to about 12 months (52 weeks) (e.g., about 2 months, about 3 months, about 4 months, about 5 months, about 6 months, about 7 months, about 8 months, about 9 months, about 10 months, or about 11 months).

It may be advantageous to administer multiple doses of the antibody or antigen binding fragment at a regular interval, depending on the therapeutic regimen selected for a particular subject. In some embodiments, the antibody or fragment thereof is administered periodically over a time period of one year (12 months, 52 weeks) or less (e.g., 9 months or less, 6 months or less, or 3 months or less). In this regard, the antibody or fragment thereof is administered to the human once every about 3 days, or about 7 days, or 2 weeks, or 3 weeks, or 4 weeks, or 5 weeks, or 6 weeks, or 7 weeks, or 8 weeks, or 9 weeks, or 10 weeks, or 11 weeks, or 12 weeks, or 13 weeks, or 14 weeks, or 15 weeks, or 16 weeks, or 17 weeks, or 18 weeks, or 19 weeks, or 20 weeks, or 21 weeks, or 22 weeks, or 23 weeks, or 6 months, or 12 months.

In various embodiments, one or more doses comprising from about 50 milligrams to about 1,000 milligrams of the antibody or antigen binding fragment thereof are administered to a subject (e.g., a human subject). For example, a dose can comprise at least about 5 mg, at least about 15 mg, at least about 25 mg, at least about 50 mg, at least about 60 mg, at least about 70 mg, at least about 80 mg, at least about 90 mg, at least about 100 mg, at least about 120 mg, at least about 150 mg, at least about 200 mg, at least about 210 mg, at least about 240 mg, at least about 250 mg, at least about 280 mg, at least about 300 mg, at least about 350 mg, at least about 400 mg, at least about 420 mg, at least about 450 mg, at least about 500 mg, at least about 550 mg, at least about 600 mg, at least about 650 mg, at least about 700 mg, at least about 750 mg, at least about 800 mg, at least about 850 mg, at least about 900 mg, at least about 950 mg or up to about 1,000 mg of antibody. Ranges between any and all of these endpoints are also contemplated, e.g., about 50 mg to about 80 mg, about 70 mg to about 140 mg, about 70 mg to about 270 mg, about 75 mg to about 100 mg, about 100 mg to about 150 mg, about 140 mg to about 210 mg, or about 150 mg to about 200 mg, or about 180 mg to about 270 mg. The dose is administered at any interval, such as multiple times a week (e.g., twice or three times per week), once a week, once every two weeks, once every three weeks, or once every four weeks.

In some embodiments, the one or more doses can comprise between about 0.1 to about 50 milligrams (e.g., between about 5 and about 50 milligrams), or about 1 to about 100 milligrams, of antibody (or antigen binding fragment thereof) per kilogram of subject body weight (mg/kg). For example, the dose may comprise at least about 0.1 mg/kg, at least about 0.5 mg/kg, at least about 1 mg/kg, at least about 2 mg/kg, at least about 3 mg/kg, at least about 4 mg/kg, at least about 5 mg/kg, at least about 6 mg/kg, at least about 7 mg/kg, at least about 8 mg/kg, at least about 9 mg/kg, at least about 10 mg/kg, at least about 11 mg/kg, at least 12 mg/kg, at least 13 mg/kg, at least 14 mg/kg, at least about 15 mg/kg, at least 16 mg/kg, at least 17 mg/kg, at least 18 mg/kg, at least 19 mg/kg, at least about 20 mg/kg, at least 21 mg/kg, at least 22 mg/kg, at least 23 mg/kg, at least 24 mg/kg, at least about 25 mg/kg, at least about 26 mg/kg, at least about 27 mg/kg, at least about 28 mg/kg, at least about 29 mg/kg, at least about 30 mg/kg, at least about 31 mg/kg, at least about 32 mg/kg, at least about 33 mg/kg, at least about 34 mg/kg, at least about 35 mg/kg, at least about 36 mg/kg, at least about 37 mg/kg, at least about 38 mg/kg, at least about 39 mg/kg, at least about 40 mg/kg, at least about 41 mg/kg, at least about 42 mg/kg, at least about 43 mg/kg, at least about 44 mg/kg, at least about 45 mg/kg, at least about 46 mg/kg, at least about 47 mg/kg, at least about 48 mg/kg, at least about 49 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 85 mg/kg, at least about 90 mg/kg, at least about 95 mg/kg, or up to about 100 mg/kg. Ranges between any and all of these endpoints are also contemplated, e.g., about 1 mg/kg to about 3 mg/kg, about 1 mg/kg to about 5 mg/kg, about 1 mg/kg to about 8 mg/kg, about 3 mg/kg to about 8 mg/kg, about 1 mg/kg to about 10 mg/kg, about 1 mg/kg to about 20 mg/kg, about 1 mg/kg to about 40 mg/kg, about 5 mg/kg to about 30 mg/kg, or about 5 mg/kg to about 20 mg/kg.

### Methods of Treatment

In another aspect, described herein is a method of treating a subject suffering from long QT syndrome (LQTS) comprising administering the antibody (or antigen binding fragment thereof) or pharmaceutical composition to the subject in an amount effective to treat long QT syndrome.

In some embodiments, the long QT syndrome is LQTS2 or LQTS3. In some embodiments, the long QT syndrome is LQTS2. In some embodiments, the long QT syndrome is LQTS3.

In some embodiments, the subject is also suffering from cardiomyopathy, diabetes, epilepsy or neurological comorbidities. In some embodiments, administering the antibody (or antigen binding fragment thereof) results in shorter cardiac repolarization compared to a subject that did not receive the antibody (or antigen binding fragment thereof). In some embodiments, administering the antibody (or antigen binding fragment thereof) results in the reduced incidence of ventricular tachyarrhythmias including sudden cardiac arrest compared to a subject that did not receive the antibody (or antigen binding fragment thereof).

In some embodiments, administering the antibody (or antigen binding fragment thereof) results in shorter cardiac repolarization (QT or JT interval on ECG, or variations thereof such as QT interval corrected by Bazett formula (QT/RR^{1/2}), Fridericia (QT/RR^{1/3}), Framingham (QT+0.154(1-RR)), Hodges (QT+1.75(HR-60), Rautaharju (QTx(120+HR)/180), heart rate-corrected JT (QTc-QRS)) of the subject by at least 5% (or at least 10%, or at least 15%, or at least 20%, or at least 25%, or at least 30%, or at least 35%, or at least 40%, or at least 45%, or at least 50% or more) compared to the cardiac repolarization of the subject at baseline.

In some embodiments, administering the antibody (or antigen binding fragment thereof) does not affect KCNQ1 channel expression in the subject.

In some embodiments the antibody has no detectable or minimal off-target effects, e.g. epilepsy, neuropsychiatric comorbidities, diabetes mellitus or impaired glucose tolerance, thyroid disorder.

### Combination Therapy

The anti-KCNQ1 monoclonal antibodies disclosed here can be administered alone or optionally in combination with other therapeutic agents useful for the treatment of LQTS. Thus, any active agent known to be useful in treating a condition, disease, or disorder described herein can be used in the methods of the invention, and either combined with the amino sterol compositions used in the methods of the invention, or administered separately or sequentially. Exemplary additional agents include, but are not limited to, beta-blockers such as propanolol (e.g., Inderal®), atenolol (e.g., Tenormin®), metoprolol (e.g., Metoprolol®, Lopressor®), nadolol (e.g., Corgard®), bisoprolol (e.g., Zebeta®, Monocor®); antiarrhythmics such mexiletine (e.g., Mexitil®), ranolazine (e.g., Ranexa®); calcium channel blockers such as diltiazem (e.g., Cardizem®) and verapamil (e.g., Verelan®); and digitalis derived drugs such as digoxin (e.g., Lanoxin®).

### Kits

Once a pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. The invention also provides kits for producing a single-dose administration unit. The kits of the disclosure may each contain both a first container having a dried protein and a second container having an aqueous formulation. In certain embodiments, kits containing single and multi-chambered pre-filled syringes (e.g., liquid syringes and lyosyringes) are provided.

The following Examples are provided to further illustrate aspects of the disclosure, and are not meant to constrain the disclosure to any particular application or theory of operation.

### EXAMPLES

### Example 1—Materials and Methods

Generation of anti-KCNQ1 antibody: Five Balb/c mice were immunized against the KCNQ1 channel peptide sequence (AEKDAVNESGRVEFGSYADA, SEQ ID NO: 10 (amino acids 283-302 of SEQ ID NO: 9)) coupled to the Keyhole limpet hemocyanin (KLH) carrier, using the standard protocol by ProteoGenix, Schiltigheim, France. Briefly, mice received a subcutaneous injection of 50 μg KCNQ1 peptide with complete Freud's adjuvant, followed by a weekly injection of 25 μg KCNQ1 peptide supplemented with incomplete Freud's adjuvant (IFA). After a final booster injection (50 μg KCNQ1 peptide+IFA) on the 4th week, spleen cells from mice with the highest antibody titer were collected and fused with myeloma cells by polyethylene glycol (ProteoGenix, Schiltigheim, France). Hybridoma cells were cultured in complete medium containing RPMI 1640/1% L-glutamine supplemented with 10% fetal bovine serum and 1% Penicillin/Streptomycin. Sequential separation of cells of different passages was performed using Hypoxanthine-Aminopterin-Thymidine (HAT) and complete medium. Through repetitive subcloning by the limiting dilution technique and screening via enzyme-linked immunosorbent assay (ELISA), six clones were identified which specifically produce IgGs targeting the KCNQ1 channel sequence. Six monoclonal antibodies were produced from the selected clones and purified on protein G columns (ProteoGenix, Schiltigheim, France). A standard ELISA was used to determine the IgG subtype. Monoclonal antibodies were collected in sterile 1× PBS (pH 7.4) and concentration was determined with the A₂₈₀ method.

Production of a recombinant antibody: The sequences of the variable regions fused to the constant region of murine IgG2a were chemically synthetized by ProteoGenix (France), in combination with signal peptides optimized for mammalian expression. The cDNAs were inserted into the expression vector pTXs 1 using the restriction enzymes, EcoRI and NotI. With Proteogenix' propriety method, the plasmid was then transiently transfected in XtenCHO™ cells. Antibodies were then affinity-purified from the supernatant of XtenCHO™ cells. After separation by SDS-PAGE gel, the monoclonal antibody was analyzed by western blot under reducing and non-reducing conditions.

Antibody kinetics and affinity measurement: A Biacore 8K Surface Plasmon Resonance (SPR) instrument (GE Healthcare Life Sciences, ProteoGenix, France) equipped with a CM5 sensor chip was used to generate binding kinetic rate and affinity constants at room temperature. IgG2a 8-F11-D4 (10 μg/ml) was immobilized onto the CM5 chip by amide coupling, following manufacturer's protocol. The surface chemistry was activated using 1-Ethyl-3-(3-dimethylaminopropyl) carbodiimide hydrochloride (EDC) and sulfo-N-Hydroxysuccinimide (NHS) reagents prior to immobilization, while ethanolamine was used to deactivate remaining active esters. Samples were prepared in HBS-EP⁺ buffer composed of 10 mM HEPES (pH 7.4), 150 mM NaCl, 3 mM EDTA, 0.05% Surfactant P20. The antigenic peptide was suspended in 20 mM sodium acetate (pH 4.5) and glycine-HCl (pH 1.5) was used as regeneration buffer. To define the binding affinity, a kinetic analysis of the interaction was performed using an antibody immobilization level of 10′000 Resonance Unit (RU). This level was considered optimal based upon the calculation of the maximal response (Rₘₐₓ) of analyte (antigenic peptide) to ligand (IgG2a): Rₘₐₓ=(MW_{analyte}/MW_{ligand})×R_{L}×Sₘ, where MW_{analyte} and MW_{ligand} are the molecular weights of the peptide (analyte, 2.12 kDa) to the antibody (ligand, 180 kDa as determined by SDS-PAGE), respectively, R_{L} is the target amount of ligand bound in RU, and Sₘ represents the stoichiometric ratio of the analyte/ligand interaction (Sₘ equals 1). A theoretical Rₘₐₓ of 100 RU is achieved at 10′000 RU of ligand. Different concentrations of the monoclonal antibody (7.8125 nM-500 nM, twofold serial dilutions) was injected at a flow rate of 30 μl/min for 120 s followed by a dissociation phase of 600 s. Injections were performed in triplicate to assess for the assay's reproducibility. All data were processed and analyzed with Biacore 8K Evaluation Software.

High-resolution conformational epitope mapping: Mapping of antibody epitopes was performed on PEPperCHIP® by PEPperPRINT GmbH, Germany, covering the full-length sequence of KCNQ1 protein (NP 000209.2) elongated with neutral GSGSGSG linkers at the C- and N-terminus to avoid truncated peptides. The elongated antigen sequence was translated into 7, 10 and 13 overlapping amino acid peptides, cyclized via a thioether linkage between a C-terminal cysteine and an appropriately modified N-terminus. The resulting conformational KCNQ1 peptide microarrays contained 2043 different peptides printed in duplicate and were framed by additional hemagglutinin (YPYDVPDYAG (SEQ ID NO: 11), 134 spots) control peptides. The monoclonal antibody was incubated at a concentration of 0.1 μg/ml. Goat anti-mouse IgG (H+L) DyLight680 served as secondary antibody. LI-COR Odyssey Imaging System was used for scanning. Quantification of spot intensities and peptide annotation were performed with PepSlide® Analyzer. Based on averaged median foreground intensities, an intensity map was generated. A maximum spot-to-spot deviation of 40% was tolerated, otherwise the corresponding intensity value was zeroed.

Molecular docking: The following three-dimensional structures were retrieved from RCSB Protein Data Bank (PDB) and served as templates to build the proteins: Homo sapiens KCNQ1-calmodulin channel complex (PDB: 6UZZ) and Mus musculus immunoglobulin 2a (PDB: 1IGT). The previously obtained IgG2a 8-F11-D4 sequence was integrated as initial data for modeling the 3D structures of the monoclonal antibody. Rigid antigen-antibody docking was performed using Molecular Operating Environment (MOE) program (Chemical Computing Group, Montreal, Canada). The antibody and the KCNQ1 channel were prepared for docking by minimizing their energy and then 3D protonation following default parameters of MOE algorithm. Target sites of both the KCNQ1 channel (extracellular domain) and the antibody (CDRs) were specified prior to docking. The loops between the transmembrane segments 51-S2, S3-S4 and S5-S6 (including the pore region) constitute the extracellular domain of KCNQ1 channel complex, as defined by Chouabe et al. (Chouabe, Neyroud, Guicheney, Lazdunski, Romey & Barhanin, 1997). The complementary-determining regions (CDRs) of the H and L chains were predicted using the Kabat numbering scheme (Kabat, Wu, Perry, Foeller & Gottesman, 1991). A large AtomQ feature (radius 20 Å) was set in the extracellular channel region, while large excluded volumes (radius 20 Å) were created at the lipid bilayer region to simulate the cell membrane. Molecular graphics and protein visualization were rendered using MOE program.

CHO cell culture and patch clamp recording: Chinese Hamster Ovary (CHO) cells stably expressing human KCNQ1/KCNE1 channels were cultured in Ham's F-12 nutrient mix (Gibco™ by Life Technologies Europe BV, Zug, Switzerland) supplemented with 10% fetal bovine serum (Bioswisstec Ltd, Schaffhausen, Switzerland), penicillin/streptomycin (penicillin 10.000 U/ml-streptomycin 10 mg/ml, Seraglob by Bioswisstec Ltd, Schaffhausen, Switzerland) at 37° C. with 5% CO₂. Cells were split enzymatically using trypsin-EDTA (Sigma Aldrich, Buchs, Switzerland). For patch clamp experiments, CHO cells were plated onto sterile Petri dishes (1000-2000 cells/cm²) in culture medium±monoclonal antibody. An EPC-10 amplifier controlled by PATCHMASTER (HEKA Elektronik GmbH, Lambrecht, Germany) was used to record I_{Ks} currents in the whole-cell configuration at room temperature. The following external solution was used (in mmol/L): 140 NaCl, 5 KCl, 1 MgCl₂, 10 HEPES, 1.8 CaCl2), 10 glucose (pH 7.4 adjusted with NaOH)±monoclonal antibody. Borosilicate glass capillaries (Harvard Apparatus, Holliston, Massachusetts, USA, tip resistances 5-7 MΩ) were filled with internal solution composed of (in mmol/L): 100 K⁺ aspartate, 20 KCl, 2 MgCl₂, 1 CaCl₂, 10 EGTA, 5 K⁺ ATP, 10 HEPES, 40 glucose (pH 7.2 adjusted with KOH). I_{Ks} currents were measured by holding the CHO cells at −60 mV and applying depolarizing test pulses (3000 ms, 0.1 Hz) from −50 mV to +70 mV in 10 mV incremental steps, followed by repolarizations (2000 ms) to −40 mV. I_{Ks} currents were low-pass filtered at 2.9 kHz and sampled at 4 kHz. Whole-cell patch clamp data were analyzed with FITMASTER (HEKA Elektronik GmbH, Lambrecht, Germany). Activation and inactivation curves were fit with a Boltzmann function: I/Imax=1/(1+e(V½−Vt)/k), where V_{1/2}=half-maximal activation potential, Vt=test pulse potential, k=slope factor (Maguy et al. J Am Coll Cardiol 2020).

hiPSC-CMC culture and patch clamp recording: Human induced pluripotent stem cell-derived ventricular cardiomyocytes (hiPSC-CMCs) from Ncardia (Pluricyte©, Ncardia BV, Leiden, The Netherlands) were cultured according to manufacturer's instructions (Maguy et al., J Am Coll Cardiol 2020). hiPSC-CMCs were plated at a density of 25′000 cells/cm2 on Petri dishes coated with Corning Matrigel (growth factor reduced basement membrane matrix from VWR International GmbH, Dietikon, Switzerland) diluted 1:100 in DMEM/F-12, GlutaMAX™ supplement (Gibco™ by Life Technologies Europe BV, Zug, Switzerland). Spontaneous action potentials were recorded between day 7 and 14 post-thawing, under current-clamp conditions with EPC-10 amplifier controlled by PATCHMASTER (HEKA Elektronik GmbH, Lambrecht, Germany) at 37° C., as previously described (Maguy et al. J Am Coll Cardiol 2020). The external solution was composed of (in mmol/L): 140 NaCl, 5 KCl, 1 MgCl₂, 10 HEPES, 1.8 CaCl₂, 10 glucose (pH 7.4 adjusted with NaOH)±monoclonal antibody. To induce LQTS2 in hiPSC-CMCs pharmacologically, cells were exposed to the selective hERG blocker, 10 nM E-4031 (Alomone Labs, Israel) and recordings began after 30 min of incubation. To simulate the electrical phenotype of LQTS3, late I_{Na} current was selectively increased using the sea anemone toxin (5 nM ATX-II, Alomone Labs, Israel) after 5 min of incubation. Borosilicate glass pipettes had tip resistances of 2-4 MΩ. The internal solution contained (in mmol/L): 110 K⁺ aspartate, 20 KCL, 1 MgCl₂, 5 Mg²⁺ ATP, 0.1 Li⁺ GTP, 10 HEPES, 5 Na⁺ phosphocreatine, 0.05 EGTA (pH adjusted to 7.3 with KOH), 200 μg/ml amphotericin (AppliChem GmbH, Germany). Action potentials were analyzed with FITMASTER (HEKA Elektronik GmbH, Germany). The action potential duration (APD) was determined at 90% (APD₉₀) repolarization.

Statistical analysis: Results are shown as mean±SEM, unless otherwise stated. All data underwent Shapiro-Wilk test to assess for the normality of distribution. Statistical differences between groups with normally distributed data were determined by one-way analysis of variance followed by Tukey's multiple comparison post hoc test. For comparisons between two group means, two-tailed Student's t-test was applied to determine the statistical significance of normally distributed data. In the case of non-normal distribution, Mann-Whitney U test was used. GraphPad Prism 7 software (GraphPad software, USA) was used for statistical analyses. A p value of <0.05 was considered statistically significant.

### Example 2—Selection of Functional KCNQ1 Monoclonal Antibodies

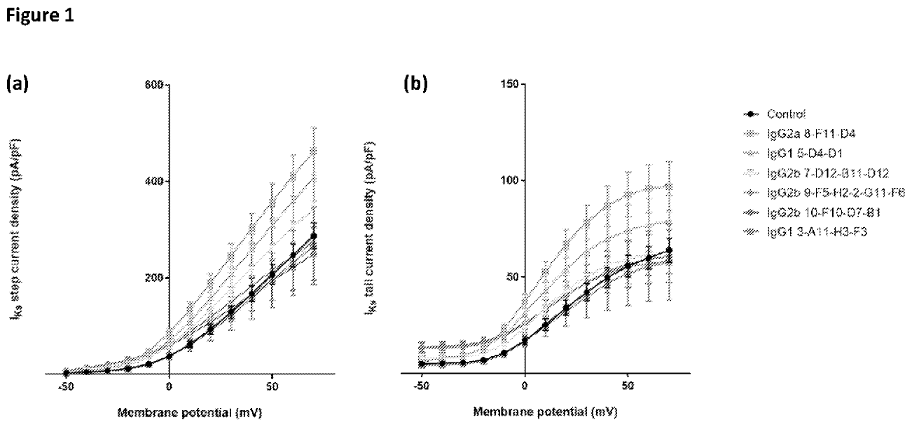

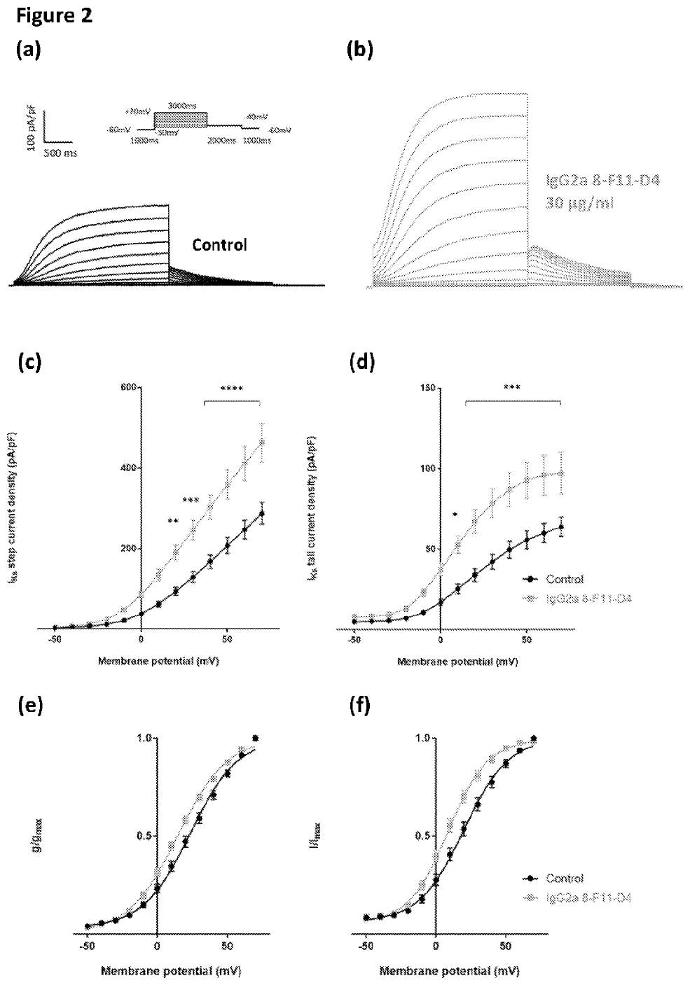

Hybridoma supernatants were tested by ELISA to ensure that the secreted antibody retained specificity for the KCNQ1 channel peptide. Out of 40 hybridoma clones, six producing functional IgG antibodies with specificity were identified (i.e., 3-A11-H3-F3; 5-D4-D1; 7-D12-B11-D12; 8-F11-D-4; 9-F5-H2-2-G11-F6 and 10-F10-D7-B1). Whole-cell patch clamp experiments were performed to study the effects of all 6 monoclonal antibodies on I_{Ks} current in CHO cells stably expressing human I_{Ks} channels. The choice to test the concentration of 30 μg/ml IgG was based on previous data on polyclonal KCNQ1 antibodies (Maguy, Kucera, Wepfer, Forest, Charpentier & Li, 2020). As illustrated in FIG. 1, IgG2a 8-F11-D4 (comprising CDRs set forth in SEQ ID NOs: 1-6) best replicated the effect of the polyclonal antibody population: IgG2a 8-F11-D4 increased the mean I_{Ks} step current by 1.6-fold at +70 mV, and the mean I_{Ks} tail current by 1.5-fold upon repolarization to −40 mV (FIGS. 2A-2D). The capacitance of CHO cells treated with monoclonal antibodies were similar to the control group. Analogous to the polyclonal KCNQ1 antibodies, IgG2a 8-F11-D4 shifted the half-maximal activation potential (V_{1/2}) by −9 mV, while shifting the voltage-dependence of deactivation to more negative potentials by 11 mV (FIG. 2E-2F). The activation and deactivation slope factors k reflecting voltage sensitivity remained unchanged.

### Example 3—Characterization of KCNQ1 Monoclonal Antibody IgG2a 8-F11-D4

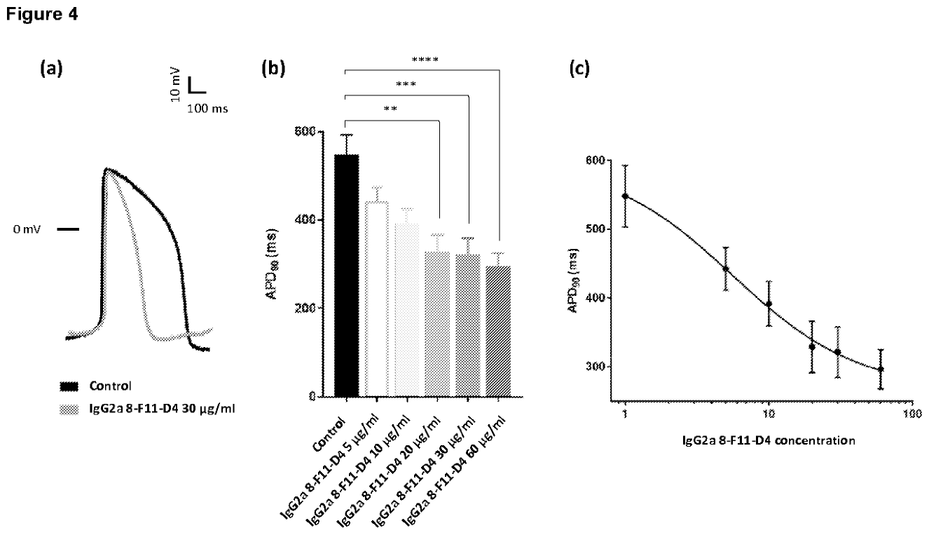

Next, the effect of IgG2a 8-F11-D4 on cardiac repolarization was tested at various concentrations (FIGS. 4A and 4B). All action potential (AP) parameters are delineated in Table 1.

| | | IgG2a 8-F11- | IgG2a 8-F11- | IgG2a 8-F11- | IgG2a 8-F11- | IgG2a 8-F11- | |
|---|---|---|---|---|---|---|---|
| | Control | D4 5 μg/ml | D4 10 μg/ml | D4 20 μg/ml | D4 30 μg/ml | D4 60 μg/ml | |
| | (n = 15) | (n = 4) | (n = 10) | (n = 11) | (n = 14) | (n = 14) | P value |
| MDP | −70.3 ± 1.7 | −62.4 ± 5.0 | −68.4 ± 1.4 | −59.1 ± 2.4 | −59.2 ± 3.1 | −55.0 ± 2.7 | 0.0001 |
| (mV) | | | | | | | |
| APA | 108.7 ± 2.7 | 98.6 ± 10.3 | 103.5 ± 3.7 | 90.21 ± 4.3 | 83.2 ± 5.9 | 78.5 ± 4.7 | <0.0001 |
| (mV) | | | | | | | |
| APD₉₀ | 548.2 ± 44.7 | 442.4 ± 30.7 | 391.6 ± 32.7 | 328.9 ± 37.5 | 321.5 ± 37.0 | 296.5 ± 28.6 | 0.0001 |
| (ms) | | | | | | | |
| Frequency | 41.2 ± 4.1 | 67.5 ± 6.3 | 57.6 ± 8.7 | 88.2 ± 10.9 | 89.7 ± 8.3 | 109.6 ± 13.1 | <0.0001 |
| (bpm) | | | | | | | |

APA = action potential amplitude,
APD = action potential duration,
MDP = maximum diastolic potential

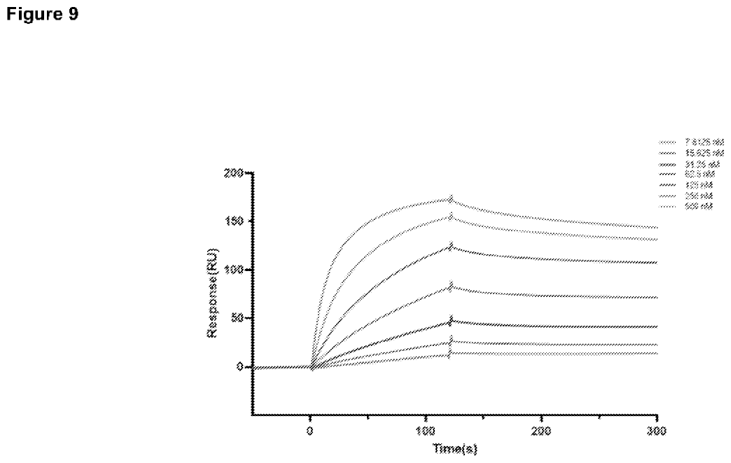

A concentration-dependent reduction of APD₉₀ by the monoclonal antibody was observed with a sigmoidal concentration-response relationship (FIG. 4C). The half-maximal effective concentration (EC₅₀) was calculated at 5.7 μg/ml IgG2a 8-F11-D4. The kinetics of monoclonal antibody binding to the respective KCNQ1 peptide was determined with SPR technique (FIG. 9). The following mean association rate constant (on rate, kₐ), mean dissociation rate constant (off rate, k_{d}) and mean equilibrium dissociation constant (K_{D}) values for the antibody-antigen interaction were calculated (±standard deviation): kₐ=9.09×10⁴±1.53×10³ M^{-s}⁻¹, k_{d}=8.20×10⁻⁴ 5.34×10⁻⁵ s⁻¹, K_{D}=9.02×10⁻⁹±6.99×10⁻¹⁰ M. The identified monoclonal antibody IgG2a 8-F11-D4 thus exhibits an overall high affinity with K_{D} values in the nanomolar range.

### Example 4—Conformational Epitope Mapping of IgG2a 8-F11-D4

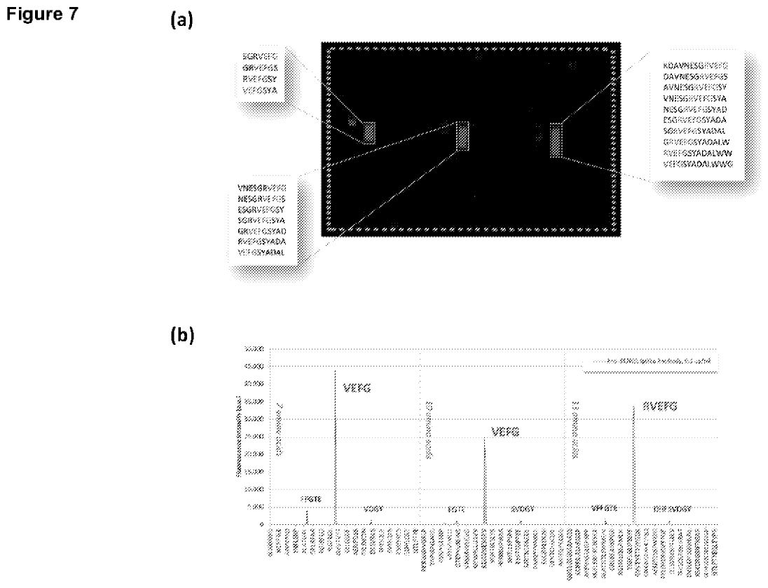

A very strong antibody response against epitope-like spot patterns formed by adjacent peptides with the consensus motif VEFG (a sequence corresponding to our targeted epitope, i.e. the extracellular loop between the 5^{th} and 6^{th} transmembrane domain of KCNQ1) was observed with all peptide lengths (FIG. 7). The arginine (R) amino acid preceding this sequence may contribute to antibody binding as well. Additional significantly weaker interactions were found for peptides with the consensus motif FGTE and VDGY presumably due to cross-reactions based on minor sequence homologies (underlined amino acid positions). Nonetheless, both peptide sequences are located intracellularly and are therefore not readily accessible to circulating antibodies.

### Example 5—KCNQ1-8-F11-D4 IgG2a Docking

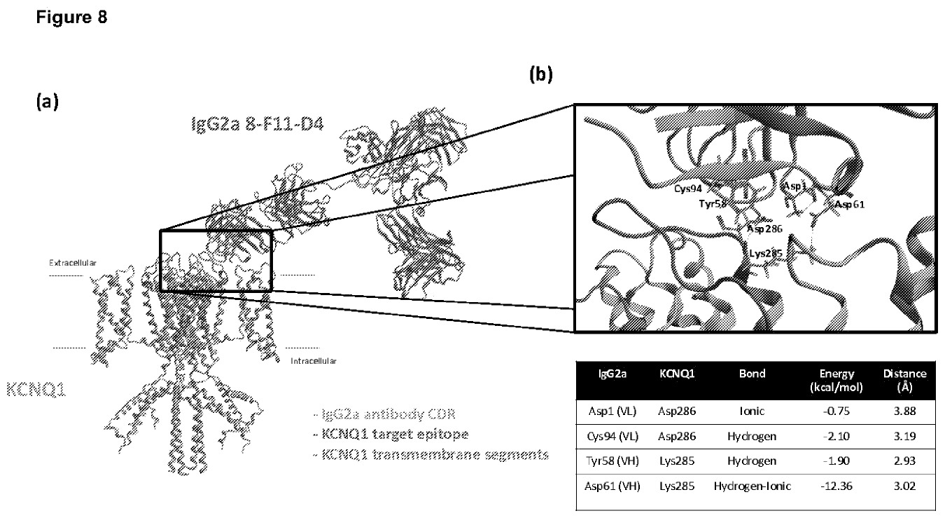

Because a peptide-based epitope mapping approach unlikely identifies involvement of tertiary and/or quaternary structure of the KCNQ1 channel, computational docking was performed. The heavy (H) and light (L) chains coding sequences of the variable region of IgG2a 8-F11-D4 were amplified from mRNA, cloned and verified by comparison with protein sequencing of affinity-purified monoclonal antibodies. The light and heavy chain variable sequences of IgG2a 8-F11-D4 are set forth in SEQ ID NOs: 7 and 8, respectively. To confirm the sequence, a recombinant murine IgG2a 8-F11-D4 antibody was developed and patch clamp measurements were performed on CHO cells stably expressing human I_{Ks}. In the presence of 30 μg/ml recombinant antibody, a similar 1.6-fold increase in I_{Ks} step current was observed compared to control cells at +70 mV, while the I_{Ks} tail current was increased by 1.5-fold. Antigen-Antibody binding sites were then searched between the surface exposed variable regions of IgG2a and the KCNQ1 channel using computational docking methods (FIG. 8A). Important residues from the light (Cys94) and heavy chains (Tyr58, Asp61) were predicted to mediate hydrogen bonding contacts to the Asp286 and Lys285 amino acids of the third extracellular loop of the channel (FIG. 8B). The Asp1 residue of the IgG light chain and Asp61 of the heavy chain form ionic interactions with Asp286 and Lys285 of KCNQ1, respectively (FIG. 8B).

### Example 6—Therapeutic Potential of IgG2a 8-F11-D4 Monoclonal Antibody In Vitro

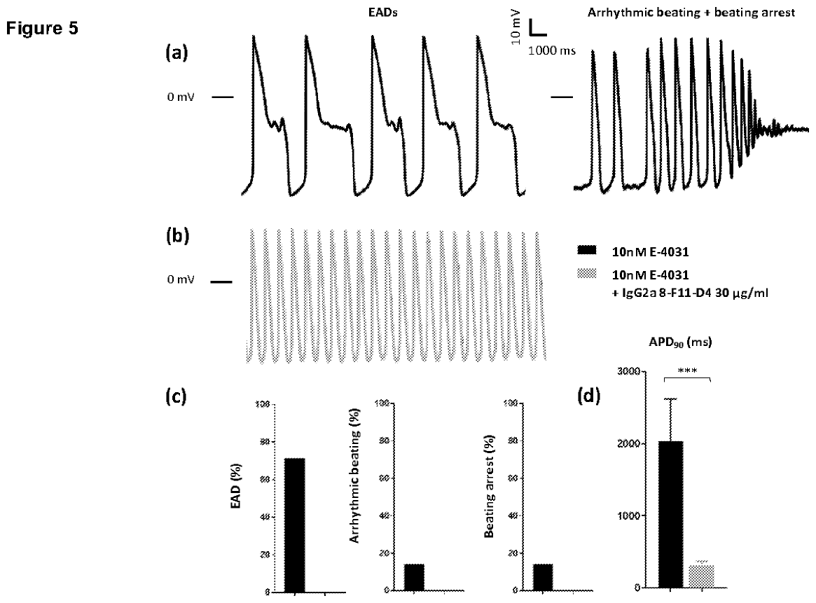

To induce LQTS type 2 pharmacologically in hiPSC-CMCs, the selective hERG inhibitor, E-4031, was used. E-4031 at 10 nM concentration resulted in a 4-fold increase in APD₉₀ (Table 2) and frequent early afterdepolarizations (EADs, 71.4%, FIG. 5A). One cell even developed arrhythmic beating that subsequently degenerated in beating arrest (FIG. 5A). IgG2a 8-F11-D4 significantly shortened APD₉₀ at 30 μg/ml concentration (FIG. 5B, Table 2). Moreover, the monoclonal antibody completely blunted arrhythmic events (FIG. 5C).

TABLE 2
Action potential characteristics of a pharmacological model of
LQTS2 in hiPSC-CMC ± IgG2a 8-F11-D4.

| | | LQTS2 | |
|---|---|---|---|
| | | hiPSC-CMC + | |
| | LQTS2 hiPSC- | IgG2a 8-F11-D4 | |
| | CMC | 30 μg/ml | |
| | (n = 7) | (n = 8) | P value |
| MDP (m V) | −60.6 ± 4.4 | −57.1 ± 2.3 | 0.485 |
| APA (m V) | 95.5 ± 9.4 | 81.5 ± 4.6 | 0.187 |
| APD90 (ms) | 2036.1 ± 587.0 | 318.9 ± 50.2 | 0.0006 |
| Frequency | 31.0 ± 4.4 | 121.9 ± 11.5 | <0.0001 |
| (bpm) | | | |

APA = action potential amplitude,
APD = action potential duration,
MDP = maximum diastolic potential

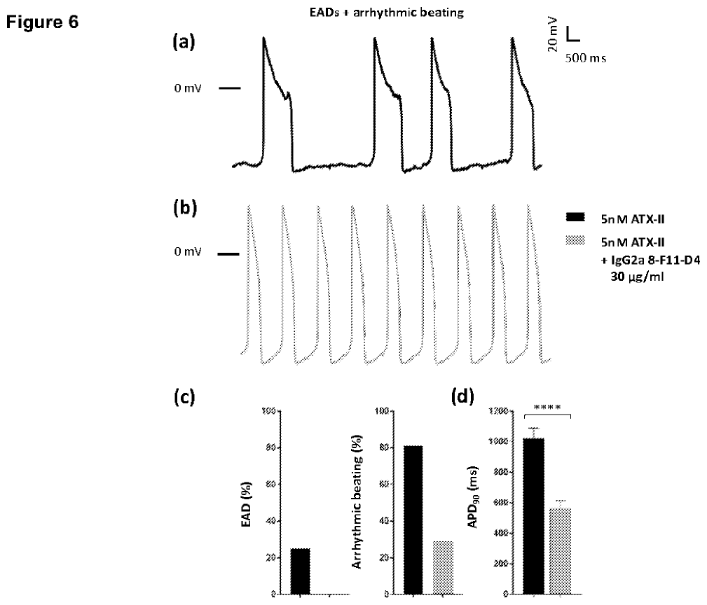

Next, the effect of IgG2a 8-F11-D4 monoclonal antibody in a cellular model of LQTS type 3 using ATX-II, that specifically enhances the late I_{Na} current (Karagueuzian, Pezhouman, Angelini & Olcese, 2017) was studied. 5 nM ATX-II prolonged the APD₉₀ by 2-fold (Table 3) and triggered EADs (25%, FIG. 6A) as well as arrhythmic beating (81.3%, FIG. 6A). When cells were treated with 30 μg/ml of IgG2a 8-F11-D4, the APD₉₀ was consistently normalized (FIG. 6B-6D, Table 3), EADs suppressed and arrhythmic beating reduced (FIG. 6C).

TABLE 3
Action potential characteristics of a pharmacological model of
LQTS3 in hiPSC-CMC ± IgG2a 8-F11-D4.

| | | LQTS3 hiPSC-CMC + | |
|---|---|---|---|
| | LQTS3 hiPSC-CMC | IgG2a 8-F11-D4 | |
| | (n = 16) | 30 μg/ml (n = 14) | P value |
| MDP (mV) | −76.3 ± 1.1 | −72.4 ± 2.3 | 0.313 |
| APA (m V) | 121.6 ± 1.1 | 111.8 ± 4.0 | 0.018 |
| APD90 | 1021.0 ± 67.2 | 564.9 ± 47.3 | <0.0001 |
| (ms) | | | |
| Frequency | 24.4 ± 3.7 | 52.4 ± 6.4 | <0.0001 |
| (bpm) | | | |

APA = action potential amplitude,
APD = action potential duration,
MDP = maximum diastolic potential

### DISCUSSION

The present study identified a murine monoclonal antibody amenable to anti-arrhythmic treatment. Collectively, IgG2a 8-F11-D4 specifically targets the extracellular pore loop to open the KCNQ1 channel. The resulting K⁺ outflow (increased I_{Ks} current) shortens the cardiac repolarization phase in hiPSC-CMCs. Its therapeutic potential was verified in a cellular model of pharmacological LQTS2. IgG2a 8-F11-D4 normalized the pathologically delayed APD and suppressed arrhythmic events. Moreover, as the first of its kind, the monoclonal antibody proved a favorable outcome in vitro in the context of LQTS3: IgG2a 8-F11-D4 shortened APD and showed anti-arrhythmic properties. Based on previous data with polyclonal antibodies, IgG2a 8-F11-D4 was tested at a concentration of 30 μg/ml (Maguy, Kucera, Wepfer, Forest, Charpentier & Li, 2020). A K_{D} of IgG2a 8-F11-D4 was measured in the nanomolar range, indicating a high binding affinity, optimal for monoclonal antibodies against membrane-bound targets (Tiwari, Abraham, Harrold, Zutshi & Singh, 2017). Both experimental and computational methods used to map the antibody-antigen interaction delineated the anticipated third extracellular domain between the fifth and sixth transmembrane segment as a specific site of target. Conformational epitope mapping demonstrated strong interactions between the antibody and the consensus motif VEFG. In contrast, the docking simulation, factoring in the tertiary and quaternary structure of KCNQ1 channels, predicted a relevance for the more proximal K and D amino acid residues. Both findings concordantly emphasize their respective importance for therapeutic efficacy in LQTS.

### Example 7—High-Content Imaging of IgG2a 8-F11-D4

The following experiment was performed to determine whether a rabbit anti-KCNQ1 (extracellular) antibody (Alomone labs) and mouse monoclonal antibody IgG2a 8-F11-D4 are capable of binding to the extracellular loop of KCNQ1 in the following cell lines:
(1) Human Kv7.1/KCNE1 (KvLQT1/minK)-CHO (tetracycline-inducible CHO cell line KCNQ1-KCNE1 linked together from Charles River laboratory). Catalog #CT6101
(2) Same as above but uninduced (as a negative control).
(3) CHO KvLQT1/MinK (KCNQ1 and KCNE1 expressed separately) cell line
(4) CHO K1-WT (an additional negative control).

Briefly, a test antibody was added to cells that were washed 2 times with ice-cold Assay Buffer and then incubated for 1-2 hours at 4° C. The cells were then washed 3 times with ice-cold Assay Buffer and the fluorophore-conjugated secondary antibody was added at the appropriate dilution in ice-cold Assay Buffer and incubated for 1 hour at 4° C. protected from light. Next, 500 nM Hoescht dye was added ice-cold PBS for final use and incubated for 5 minutes at room temperature, washed three times with ice-cold Assay Buffer and then drained. Ice-cold PBS was then added to cover the cells before proceeding with microscopy.

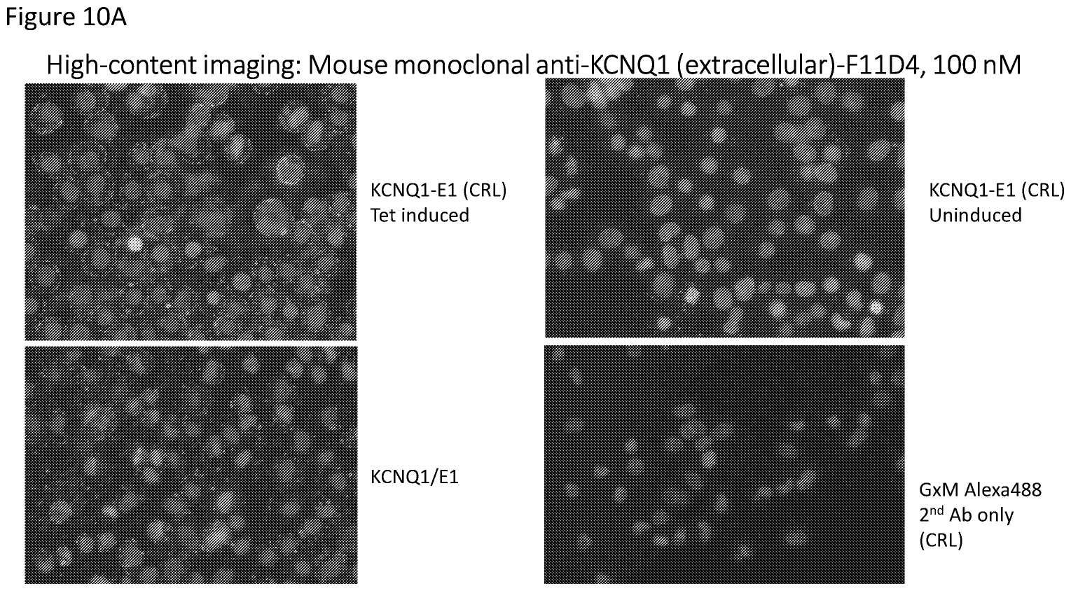

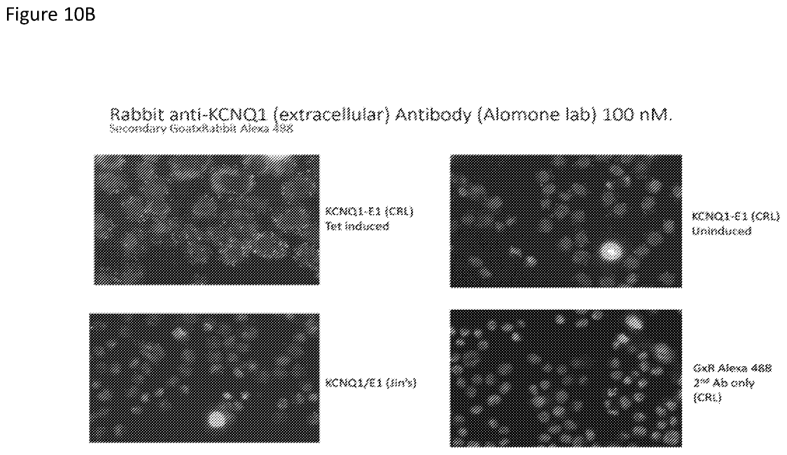

Results showed that the rabbit polyclonal KCNQ1 (extracellular) and mouse monoclonal IgG2a 8-F11-D4 antibodies bind to KCNQ1 in both the inducible and stable CHOK1 cell lines (see FIGS. 10A and 10B).

### Example 8—IgG2a 8-F11-D4 Monoclonal Antibody Shortened QT Interval In Vivo

Rabbits (male, New Zealand White) were implanted with Millar ECG telemetry electrodes in Lead 2 position and were given a minimal recovery period of 7 days. IgG2a 8-F11-D4 was formulated in 10 mM acetate, pH 5.2, 0.01% (w/v) Polysorbate 80, 9% (w/v) sucrose at 20 mg/mL. Telemetry-instrumented rabbits were treated with varying doses of IgG2a 8-F11-D4 (5 mg/kg, 10 mg/kg, 20 mg/kg and 40 mg/kg) or vehicle.

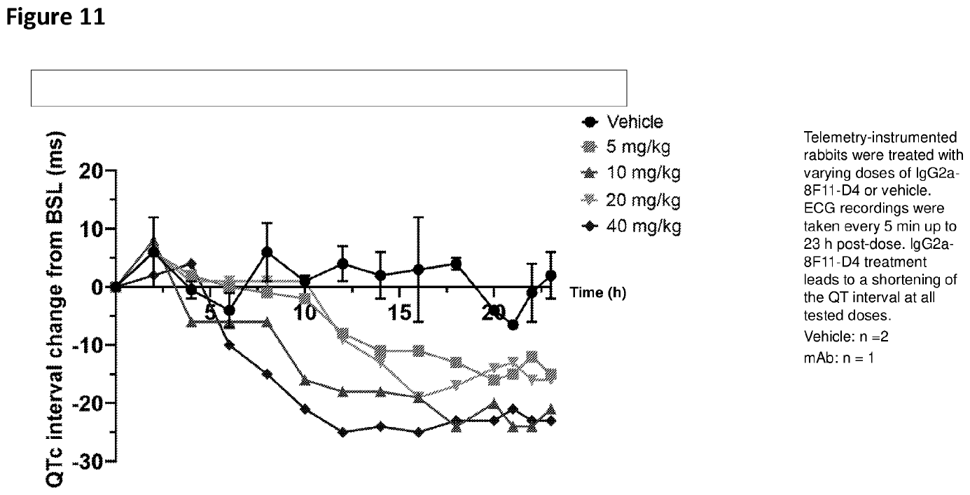

On the experimental day, ECG was recorded continuously for 5 minutes prior to intravenous injection (via the marginal ear vein) of monoclonal antibody or vehicle at 2 mL/kg. Animals were transferred in a plastic cage for continuous ECG recording with measurements being recorded every 5 minutes up to 23 h post-dose. ECG was automatically analyzed in real-time while recording. Following a steady-state shortening of the QT interval (i.e. 23 h), animals were anesthetized with ketamine/xylazine and prepared for Methoxamine+dofetilide challenge (Carlsson's model). Carlsson's challenge continued until animals progressed to TdP or 40 min of Methoxamine infusion had passed. As shown in FIG. 11, treatment with IgG2a 8-F11-D4 resulted in the shortening of the QT interval in the rabbits at all tested doses. Initial shortening of the QT interval was observed at 6-12 h post-dose, with a plateau/steady-state shortening at about 12-20 h post-dose.

### Example 9—IgG2a 8-F11-D4 Monoclonal Antibody Provided Protection Against Drug-Induced QT Prolongation in a Dose-Dependent Manner

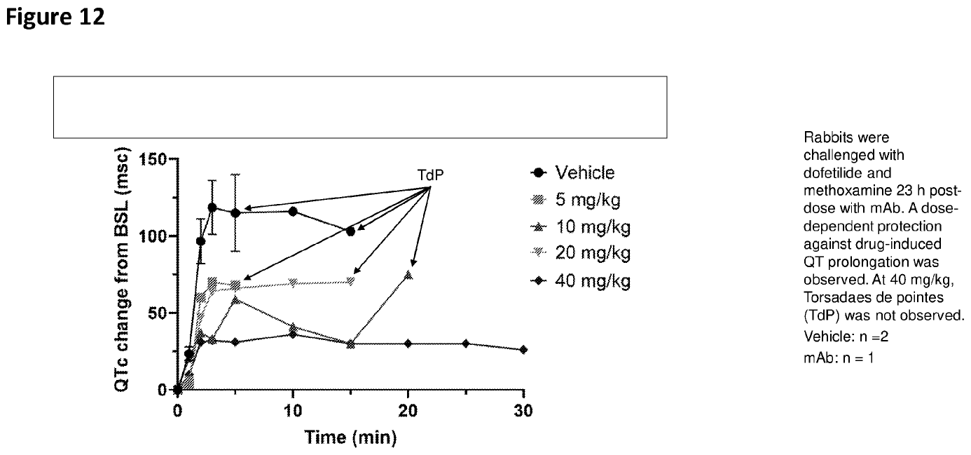

In order to determine if the anti-KCNQ1 antibodies had an effect in a model of Long QT syndrome, rabbits were challenged with dofetilide and methoxamine 23 hours after dosing with varying doses of IgG2a 8-F11-D4 (5 mg/kg, 10 mg/kg, 20 mg/kg or 40 mg/kg) or vehicle to induce longer QT intervals in the animals. ECG recordings were taken every 5 min up to 30 minutes post-dofetilide infusion. As shown in FIG. 12, IgG2a 8-F11-D4 treatment protects against drug-induced QT prolongation and arrhythmias. Carlsson model induction was performed at 23 h post-dose. A dose-dependent protection against drug-induced QT prolongation was observed. At the highest dose (40 mg/kg), the QT interval post-induction of the Carlsson model was 100 ms shorter than vehicle. The rabbit treated at this dose did not experience torsades de pointes.

### Example 10—Functional Characterization of IgG2a 8-F11-D4

The objective of this work was to determine the effects of test antibodies on the I_{Ks} channel complex (KCNQ1+KCNE1) in mammalian cells using the manual patch clamp technique.

tsA201 cells (transformed human embryonic kidney 293 cells) were grown in modified Eagle's medium (MEM) supplemented with 10% fetal calf serum and 100 g/ml penicillin, 100 g/ml streptomycin, and 0.25 100 μg/ml amphotorecin. TsA201 cells are HEK293 cells stably transfected with the SV40 large tumor antigen allowing higher level of expression of vectors containing the SV40 vector such as pCDNA3.1 used in the constructs. Cells were maintained at 37° C. in an air/5% CO₂ incubator. The day before transfection, cells were washed with MEM, treated with trypsin/EGTA for one minute and plated on 25 mm coverslips. KCNQ1, KCNE1 and GFP cDNAs were transfected using lipofectamine 2000.

Green Fluorescent Protein cDNA (GFP, 1 g) was co-transfected along with KCNQ1 and KCNE1 to identify transfected cells. Antibody treatment was started 24 h after transfection, for a 24 h period. Electrophysiology procedures: Coverslips containing cells were removed from the incubator and placed in a superfusion chamber (volume 250 μl) containing the external bath solution and maintained at room temperature. Test antibody (30 ug/mL) was present in the external solution throughout the experiment. Whole-cell current recordings were performed using an Axopatch 200B amplifier. Patch electrodes were pulled from thin-walled borosilicate glass on a horizontal micropipette puller and fire-polished. Electrodes had resistances of 1.5-3.0 mW when filled with control filling solution. Analog capacity compensation and 60%-85% series resistance compensation was used in all measurements. Data were sampled at 10-20 kHz and filtered at 5 to 10 kHz before digitization and stored on a computer for later analysis using pClamp10 software. A current-voltage (I-V) protocol consisting of a 4-second step protocol with pulses from −80 mV to +80 mV, followed by a repolarizing step to −40 mV for 2 seconds was applied. Holding potential was −90 mV and interpulse interval was 15 seconds.

### Results

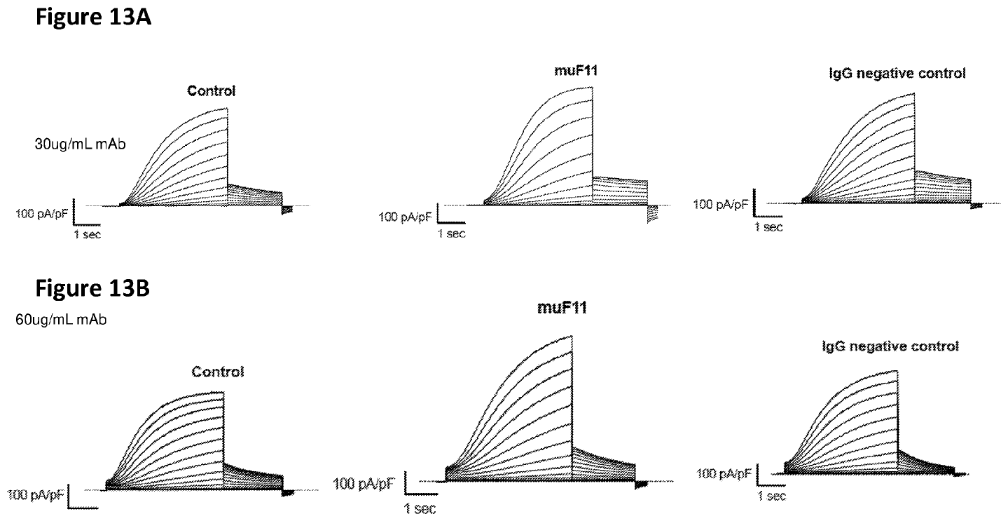

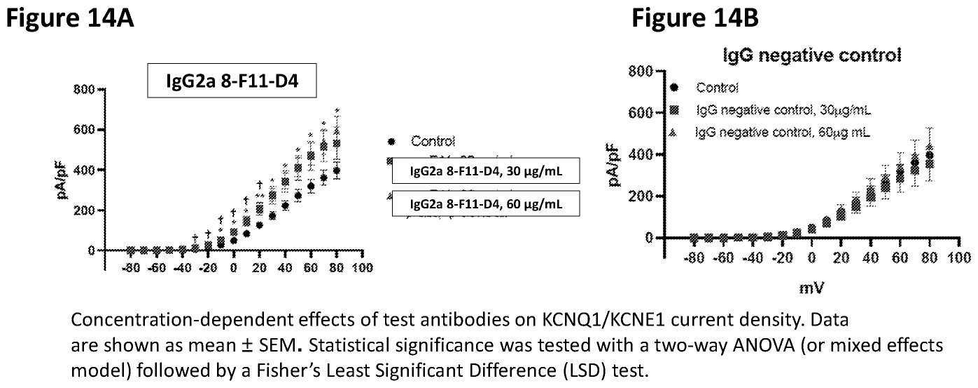

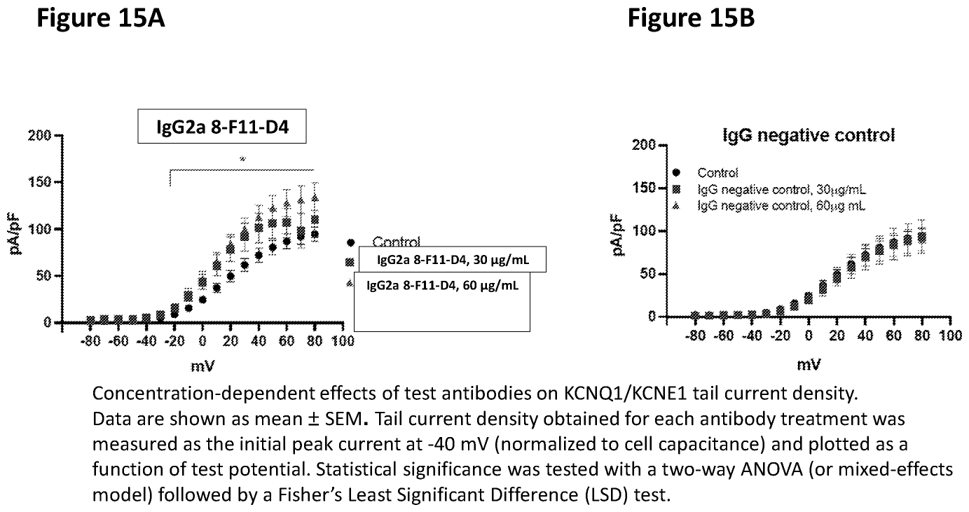

Current density was increased in the presence of IgG2a 8-F11-D4 at a concentration of 30 ug/mL (FIG. 13A) and 60 ug/mL (FIG. 13B). In particular, the I_{Ks} step and tail current densities were increased at membrane potentials more positive than −20 mV at both 30 and 60 ug/mL IgG2a 8-F11-D4 (FIGS. 14 and 15). Importantly, the increase in current density at membrane potentials more positive than −20 mV corresponds to the physiologically relevant potentials at which I_{Ks} channel is open (Jesperson et al., Physiology 20:408-416, 2005). At both 30 and 60 ug/mL, IgG2a 8-F11-D4 significantly shifted the voltage-dependence of activation to more negative potentials (Tables 4 and 5, respectively).

TABLE 4
Activation V_{1/2} and slope value (k-factor) in the absence (control)
and in the presence of 30 μg/mL of test antibodies.

| Antibody | V ½ 9 mV) ± SD | k-factor 9 mV) ± SD | n |
|---|---|---|---|
| Control | 24.4 ± 5.9 | 17.0 ± 1.5 | 14 |
| IgG2a 8-F11-D4 | 9.6 ± 10.2* | 15.0 ± 3.9 | 10 |

*p < 0.05 vs. control

TABLE 5
Activation V_{1/2} and slope value (k-factor) in the absence (control)
and in the presence of 60 μg/mL of test antibodies.

| Antibody | V ½ 9 mV) + SD | k-factor 9 mV) + SD | n |
|---|---|---|---|
| Control | 24.5 ± 0.2 | 17.9 ± 2.1 | 22 |
| IgG2a 8-F11-D4 | 11.3±* | 14.4 ± 2.7 | 9 |

*p < 0.05 vs. control

Conclusions: IgG2a 8-F11-D4 increased K⁺ outflow in HEK293 cells transiently transfected to express the human I_{Ks} channel.

### Example 11—Binding Affinity of IgG2a 8-F11-D4

The following experiment was performed in order to determine the affinity of the murine monoclonal anti-KCNQ1 monoclonal antibody IgG2a 8-F11-D4 to a target peptide that corresponds to the extracellular loop region of the KCNQ1 channel protein.

Binding kinetics of KCNQ1 peptide interactions with IgG2a 8-F11-D4 was monitored using Octet RED96 Bio-Layer Interferometry (Sartorius). All measurements were performed in duplicate at 25° C. in an assay-specific buffer containing 1× PBS pH 7.4, 1× kinetic buffer and 1% BSA using 96-well plates with orbital shake speed of 1,000 rpm. The binding curves were generated by first immobilizing 1 mM N-terminally biotinylated KCNQ1 peptide on streptavidin-coated biosensor tips for 2.5 min followed by a baseline equilibration step for 5 min. The biosensor tips were then submerged in wells containing 100 mM mAbs for 5 min to monitor formation of the mAb-peptide complex, followed by antibody dissociation in the assay buffer for 5 minutes. A shift in the interference pattern of white light reflected from the surface of a biosensor tip caused by antibody binding/dissociation was monitored in real time. Antibody-peptide interactions were analyzed by a 1:1 binding model and the dissociation constants (KDs) were determined as ratios of dissociation (koff) and association (kon) binding rate constants derived using non-linear fitting model in GraphPad Prism.

### Results

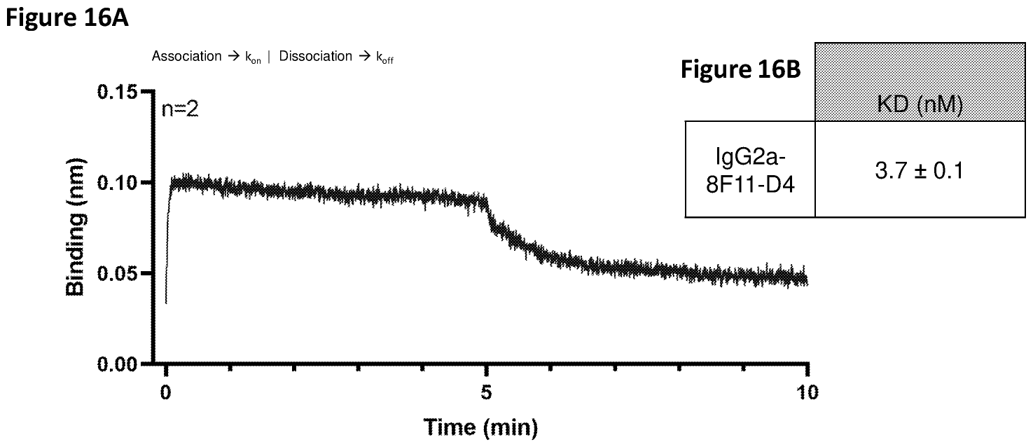

IgG2a 8-F11-D4 interacted with the KCNQ1 peptide with high affinity (K_{D}=˜4 nM). See FIG. 16.

### REFERENCES

1. Schwartz et al., Circ Arrhythm Electrophysiol 2012; 5:868-877.
2. Wu et al., Card Electrophysiol Clin. 2016; 8:275-284.
3. Schwartz et al., Eur Heart J. 2013; 34:3109-3116.
4. Li et al., Cardiovasc Res. 2013; 98:496-503.
5. Li et al., Heart Rhythm. 2014; 11:2092-2100.
6. Restier et al., J Physiol. 2008; 586:4179-4191.
7. Salata et al., Mol Pharmacol. 1998; 54:220-230.
8. Werry et al., Proc Natl Acad Sci USA. 2013; 110:E996-1005.
9. Sesti et al., J Gen Physiol. 1998; 112:651-663.
10. Crotti et al., Orphanet J Rare Dis. 2008; 3:18.
11. Priori et al., JAMA, 2004; 292:1341-1344.
12. Itoh et al., J Hum Genet. 2001; 46:38-40.
13. Migdalovich et al., Heart Rhythm. 2011; 8:1537-1543.
14. Poterucha et al., Heart Rhythm. 2015; 12:1815-1819.
15. Webster et al., Circ Arrhythm Electrophysiol. 2015; 8:1007-1009.
16. Abbott G., New J Sci2014; 2014:1-26.
17. Lazzerini et al., Nat Rev Cardiol. 2017; 14:521-535.

What is claimed is:

1. An isolated monoclonal antibody or antigen-binding fragment thereof that specifically binds human KCNQ1, the antibody comprising a set of 6 CDRs set forth in SEQ ID NOs: 1-6.

2. The antibody of claim 1, comprising a light chain variable region amino acid sequence set forth in SEQ ID NO: 7.

3. The antibody of claim 1 or claim 2, comprising a heavy chain variable region amino acid sequence set forth in SEQ ID NO: 8.

4. The antibody of claim 1, wherein the antigen binding fragment is a Fab fragment or an scFv.

5. The antibody of any one of claims 1-4, further comprising a heavy chain constant domain.

6. The antibody of any one of claims 1-5, comprising a light chain constant domain.

7. A pharmaceutical composition comprising the antibody of any one of claims 1-6 and a pharmaceutically acceptable carrier, diluent or excipient.

8. A nucleic acid comprising a nucleotide sequence that encodes the heavy chain variable region and/or light chain variable region of the antibody of any one of claims 1-6.

9. A vector comprising the nucleic acid of claim 8.

10. A host cell comprising the nucleic acid of claim 8 or vector of claim 9.

11. The host cell of claim 10 that is an eukaryotic cell.

12. The host cell of claim 11, wherein the eukaryotic cell is a CHO cell, or a human embryonic kidney 293 (HEK293) cell.

13. A method of treating a subject suffering from long QT syndrome (LQTS) comprising administering the antibody of any one of claims 1-6 to the subject in an amount effective to treat long QT syndrome.

14. The method of claim 13, wherein the long QT syndrome is LQTS1, LQTS2 or LQTS3.

15. The method of claim 13 or claim 14, further comprising administering a standard of care to the subject for the treatment of long QT syndrome.

16. The method of claim 15, wherein the standard of care is a beta-blocker, an implantable cardioverter-defibrillator (ICD), or a left cardiac sympathetic denervation.

17. The method of any one of claims 13-16, wherein the subject is also suffering from cardiomyopathy, diabetes, epilepsy or neurological comorbidities.

18. The method of any one of claims 13-17, wherein administering the antibody results in shorter cardiac repolarization compared to a subject that did not receive the antibody.

19. The method of any one of claims 13-18, wherein administering the antibody results in the reduced incidence of ventricular tachyarrhythmias including sudden cardiac arrest compared to a subject that did not receive the antibody.

20. The method of any one of claims 13-18, wherein administering the antibody does not affect KCNQ1 channel expression in the subject.
